(19) United States
(12) Patent Application Publication
(10) Pub. No.: US 2021/0378529 A1
(43) Pub. Date: Dec. 9, 2021

(54) Non-Invasive Blood Pressure Monitor

(71) Applicant: The Trustees of Columbia University in the City of New York, New York, NY (US)

(72) Inventors: David COLBURN, New York, NY (US); Samuel K. SIA, New York, NY (US)

(73) Assignee: The Trustees of Columbia University in the City of New York, New York, NY (US)

(21) Appl. No.: 17/400,485

(22) Filed: Aug. 12, 2021

Related U.S. Application Data
(63) Continuation-in-part of application No. 17/204,352, filed on Mar. 17, 2021.
Application No. 17/204,352 is a continuation of application No. PCT/US2019/051431, filed on Sep. 17, 2019.
(60) Provisional application No. 62/840,969, filed on Apr. 30, 2019.
(60) Provisional application No. 62/779,690, filed on Dec. 14, 2018.
(60) Provisional application No. 62/734,573, filed on Sep. 21, 2018.

(51) Int. Cl. A61B 5/021; A61B 5/00

(52) U.S. Cl. CPC A61B 5/02125; A61B 5/7267; A61B 5/725; A61B 2562/0223; A61B 2560/0257; A61B 2562/0219; A61B 2560/0223; A61B 5/7278

(57) ABSTRACT

The disclosed subject matter includes a wearable device for blood pressure monitoring. The embodiments employ a set of sensors to calculate a relative external pressure. A transmural pressure error can be calculated based on the relative external pressure. A measured transmural pressure can be corrected based on the transmural pressure error. Some embodiments track altitude to calculate relative external pressure error. Some embodiments track arm orientation to calculate relative external pressure error.

### CROSS-REFERENCE TO RELATED APPLICATIONS

This application is a continuation-in-part of U.S. patent application Ser. No. 17/204,352, filed Mar. 17, 2021, which is a continuation of International Patent Application No. PCT/US2019/051431 filed Sep. 17, 2019, which claims priority to U.S. Provisional Application No. 62/734,573 filed Sep. 21, 2018, U.S. Provisional Application No. 62/779,690 filed Dec. 14, 2018, and U.S. Provisional Application No. 62/840,969 filed Apr. 30, 2019, each of which is hereby incorporated by reference in its entirety.

### STATEMENT REGARDING FEDERALLY SPONSORED RESEARCH OR DEVELOPMENT

This invention was made with government support under 1UL1TR001873-01 awarded by the National Institutes of Health and 1644869 awarded by the National Science Foundation. The government has certain rights in the invention.

### BACKGROUND

Smart and connected health is a potentially transformative method for predicting early onset of disease that can advance healthcare from reactive to proactive and shift the focus from disease to well-being. However, a major roadblock to achieving this vision is the dearth of user-friendly devices that can track meaningful health data that are accurate, minimally invasive, and unobtrusive. Blood pressure (BP) monitoring is known to provide deep insights into a patient's health for a variety of conditions, including infectious and chronic diseases. Cuffless monitoring can be a desirable type of BP monitoring in certain circumstances.

### SUMMARY

Embodiments of the disclosed subject matter include a wearable device for cuffless blood pressure monitoring that does not require external per-person calibration, such as with a cuff-based measurement device. Rather, the embodiments can self-calibrate to ensure accurate blood pressure readings. Other embodiments of the device can include a cuff for blood pressure monitoring and/or improved calibration techniques.

Embodiments may include five distinct components to enhance the accuracy of cuffless BP monitoring: (1) a pulse wave detection system, (2) an external pressure compensation system, (3) a processing unit and algorithm for blood pressure tracking, (4) a processing unit and algorithm for calibration, and (5) a processing unit and algorithm for detecting periods of stable blood pressure.

One aspect of the invention is directed to a first cuffless blood pressure monitor. The first monitor comprises a signal acquisition element including a set of sensors that generate data responsive to transmural and relative external pressure. The sensors including at least two of a barometer, gyroscope, and an accelerometer. The first monitor also comprises a processor configured to track altitude and calculate relative external pressure responsively to signals from two or more of the barometer, the gyroscope, and the accelerometer, and output the relative external pressure. The processor is further configured to calculate a transmural pressure responsively to a signal from at least one pulse wave sensor based on the relative external pressure.

Some embodiments of the first monitor further comprise the at least one pulse wave sensor. The at least one pulse wave sensor includes at least one plethysmograph sensor and at least one of (a) a second plethysmograph sensor, wherein the two plethysmograph sensors can be used to measure pulse transit time or pulse wave velocity, and (b) a sensor that detects heartbeat that can be used to estimate pulse transit time or pulse wave velocity.

Some embodiments of the first monitor further comprise a magnetometer, and the processor is configured to track altitude and calculate relative external pressure responsively to signals from the magnetometer as well as the barometer, gyroscope, and accelerometer.

Another aspect of the invention is directed to a second cuffless blood pressure monitor. The second monitor comprises a device support that can be worn over an artery. The device support has a pulse wave detection element, an external-pressure processing element, a blood pressure tracking processing element, a calibration processing element, and a stability processing element, wherein the stability processing element is configured to detect periods of stable blood pressure. The pulse wave detection element includes at least one plethysmographic sensor which outputs a wave form. The external-pressure processing element includes a processor to estimate external pressure from both of a contact pressure sensor for measuring contact pressure when applied to a user and a hydrostatic pressure sensor that includes two or more of an accelerometer, a gyroscope, and a barometer, wherein the external-pressure processing element is configured to combine signals from the two or more of an accelerometer, a gyroscope, a barometer to track altitude changes in real-time.

In some embodiments of the second monitor, the external-pressure processing element includes the hydrostatic pressure sensor and is configured to combine signals from the two or more of an accelerometer, a gyroscope, a barometer, with signals from a magnetometer to track the altitude changes in real-time. Optionally, in these embodiments, the hydrostatic pressure sensor includes all three of an accelerometer, a gyroscope, and a barometer.

In some embodiments of the second monitor, the at least one plethysmographic sensor is one plethysmographic sensor, and the pulse wave detection element also includes a sensor that detects a subject's heartbeat.

In some embodiments of the second monitor, a relationship between blood pressure and the signals from the sensors is obtained by an analytical algorithm, a linear regression, a polynomial regression, machine learning, or a combination thereof. Optionally, in these embodiments, the blood pressure and the sensors are related by monitoring the change in external pressure over a predefined period of time and the effect on the signals acquired by the sensors such that blood pressure is constant over the predefined period of time so that the calibration processing element can calculate parameters needed to fit or update the algorithm used for blood pressure tracking.

Optionally, in the embodiments described in the previous paragraph, the relationship between blood pressure and the sensors is obtained when the stability processing element indicates blood pressure is constant over the predefined period of time. Optionally, in the embodiments described in the previous paragraph, a calibration is automatically begun in response to a change in external pressure and the calibration processing element outputs instructions on a display indicating steps for a user-assisted calibration.

In some embodiments of the second monitor, the wave form is used to obtain pulse wave velocity, transmural pressure, or blood pressure using an empirical algorithm, and the controller is configured to output a signal indicating an estimate of blood pressure.

Another aspect of the invention is directed to a third blood pressure monitor. The third monitor comprises a set of sensors including at least an accelerometer and a pulse wave sensor; and a processor configured to track arm orientation based on signals from the accelerometer and, based on the tracked arm orientation, calculate a transmural pressure based on signals from the pulse wave sensor.

Some embodiments of the third monitor further comprise a signal acquisition element including a set of sensors that generate data responsive to transmural and relative external pressure.

In some embodiments of the third monitor, the set of sensors further includes at least one of a gyroscope and a magnetometer.

In some embodiments of the third monitor, the processor is configured to track arm orientation using a trained deep neural network. Optionally, in these embodiments, the trained deep neural network comprises at least one bi-directional Long Short-Term Memory (LSTM) layer.

In some embodiments of the third monitor, the processor is configured to track arm orientation by feeding output signals from the set of sensors into an Unscented Kalman Filter. In some embodiments of the third monitor, the set of sensors includes a time-of-flight sensor. In some embodiments of the third monitor, a person specific calibration is performed to configure the processor to track the arm orientation.

In some embodiments of the third monitor, the processor is configured to calculate a transmural pressure by computing a transmural pressure error based on the tracked arm orientation and compensating for the transmural pressure error. In some embodiments of the third monitor, the processor is configured to track arm orientation based on signals from the accelerometer, and the accelerometer is located at a user's wrist.

Although the components are listed separately, it should be clear they may be embodied in a same processing unit. For example, all three processing units may be embodied in a single processor or computer.

### BRIEF DESCRIPTION OF THE DRAWINGS

Embodiments will hereinafter be described in detail below with reference to the accompanying drawings, wherein like reference numerals represent like elements. The accompanying drawings have not necessarily been drawn to scale. Where applicable, some features may not be illustrated to assist in the description of underlying features.

FIG. 1 illustrates PWV acquisition and conversion to blood pressure.

FIG. 2 illustrates a design for a pulse wave detection system that uses photoplethysmography.

FIG. 3 is a photo photoplethysmographic sensor used on the finger.

FIG. 4 illustrates a graph of example data from the device of FIG. 3.

FIG. 5 illustrates a graph of the data of FIG. 4 after being filtered.

FIG. 6 illustrates a graph of data the demonstrates how pulse wave velocity is calculated based on the data of FIG. 5.

FIG. 7 illustrates a block diagram of the sensor fusion algorithm used for altitude tracking.

FIG. 8 illustrates an example implementation of the hydrostatic pressure compensator.

FIG. 9 illustrates data demonstrating the effectiveness of the sensor fusion technique.

FIG. 10 illustrates a graph of a filtered photoplethysmography signal and parameterized features.

FIG. 11 illustrates a graph of filtered altitude data and parameterized features.

FIG. 12 illustrates a diagram that demonstrates how relative altitude can be related to path length traveled as shown in Equation 17.

FIG. 13 illustrates a block diagram demonstrating use of parameterized features in blood pressure estimation.

FIGS. 14-16 illustrate graphs of data that show how an external pressure compensation unit can track hydrostatic effects.

FIGS. 17-19 illustrate graphs of data that how external pressure compensation can improve systolic pressure estimation accuracy.

FIG. 20 illustrates a deep learning model in accordance with some embodiments.

FIG. 21 illustrates a graph of time series data of predicted versus measured upper arm orientation.

FIG. 22 illustrates a block diagram of an algorithm used to estimate arm orientation that takes a non-autoregressive approach.

FIG. 23 illustrates a block diagram of an algorithm with a time-of-flight sensor used to estimate arm orientation that takes an autoregressive approach.

FIG. 24 illustrates a block diagram of an algorithm used to estimate arm orientation that takes a hybrid approach.

FIG. 25 illustrates an overview of approach for tracking arm orientation.

FIG. 26 illustrates techniques for tracking of arm pose from a single wrist-based IMU using parametrized arm-pose coordinate system and deep learning.

FIG. 27 illustrates diagrams that depict changes in PTT induced by hydrostatic pressure, as modeled analytically and experimentally validated.

FIG. 28 illustrates diagrams of BP with correction for hydrostatic pressure error.

FIG. 29 illustrates diagrams for intrinsic ZYX Tait-Bryan angles and IMU alignment.

FIG. 30 illustrates graphs that depict a dependence of PTT on pressure for varied parameters, in an analytical wave-propagation equation.

FIG. 31 illustrates graphs that depict a dependence of PTT on h, in an analytical wave-propagation equation.

FIG. 32 illustrates a PTT vs h estimation plot, based on data from human subjects.

FIG. 33 illustrates a time series of best-fit PTT prediction error for the uncorrected and corrected models compared to the measured reference from a representative participant.

FIG. 34 illustrates an estimation plot for best-fit PTT prediction MAE.

FIG. 35 illustrates an estimation plot for best-fit PTT prediction MAE stratified by h.

FIG. 36 illustrates PTT prediction Bland-Altman plots.

FIG. 37 illustrates repeated measures correlation for best-fit PTT prediction plots.

FIG. 38 illustrates time series BP prediction error plots.

FIG. 39 illustrates estimation plots for DBP prediction MAE.

FIG. 40 illustrates estimation plots for SBP prediction MAE.

FIG. 41 illustrates estimation plots for DBP prediction MAE stratified by h.

FIG. 42 illustrates estimation plots for SBP prediction MAE stratified by h.

FIG. 43 illustrates prediction Bland-Altman plots, with data from three heights shown separately.

FIG. 44 illustrates SBP prediction Bland-Altman plots, with data from three heights shown separately.

FIG. 45 illustrates a block diagram of an example computer system.

### DETAILED DESCRIPTION

### Section 1

Embodiments include a wearable device for blood pressure monitoring that calculates a relative external pressure to improve accuracy. Section 1 discloses a set of embodiments for blood pressure monitoring.

Some embodiments include a wearable device for cuffless blood pressure monitoring that does not require external per-person calibration, such as with a cuff-based measurement device. Rather, embodiments can self-calibrate to ensure accurate blood pressure readings.

Some embodiments may include five distinct components:

1. a pulse wave detection system,

2. an external pressure compensation system,

3. a processing unit and algorithm for blood pressure tracking,

4. a processing unit and algorithm for calibration, and

5. a processing unit and algorithm for detecting periods of stable blood pressure.

The processing units and algorithms need not be separate processors but may be handled using a single processor.

Embodiments of the pulse wave detection system described herein comprise two sensors for recording surrogate proximal and distal signals of the pulse wave, primarily for determination of pulse wave velocity. Pulse wave velocity is the velocity of the pulse wave as it travels down the arterial network and is known to be highly predictive of blood pressure, such as by Equation 1:

BP=K₁ ln(PWV)+K₂ (1)

where K₁ and K₂ are user-specific calibration coefficients. In addition to pulse wave velocity, the pulse signal is used to calculate a number of additional features related to blood pressure.

Pulse wave can be measured by various mechanisms, including any of the following or any combination of the following:

1: two plethysmograph sensors that can be used to measure pulse transit time or pulse wave velocity;

2: one plethysmograph sensor+a sensor that detects heartbeat (e.g. ECG) that can be used to estimate pulse transit time or pulse wave velocity;

3: one or more plethysmograph sensors that can be used to estimate pulse transit time or pulse wave velocity algorithmically from the shape of the waveform;

4: one or more plethysmograph sensor that can be used to estimate transmural pressure algorithmically from the shape of the waveform; or

5: Doppler ultrasound sensor that can be used to measure pulse wave velocity.

6: Magnetic resonance imaging can be used to measure pulse wave velocity and transit time.

Further the plethysmographic sensor may include any suitable configuration including on or a combination of: (1) photoplethysmographic sensor, (2) impedance plethysmographic sensor, (3) strain gauge plethysmographic sensor, (4) magnetic plethysmographic sensor (5) air-displacement plethysmographic sensor, (6) water-displacement plethysmographic sensor, (7) ultrasound based plethysmographic sensor, or (8) an alternative sensor that acquires a non-invasive signal of the pulse wave. In other embodiments, instead of using plethysmographic sensors to measure the velocity of the pulse wave, the velocity of the pulse wave can be estimated using Note that in any of embodiments can be modified to form new embodiments by replacing any recited plethysmographic sensor with a device that measures the velocity of a pulse wave by other means such as the identified Doppler ultrasound.

In an alternative implementation, the proximal signal is acquired using electrocardiogram electrodes and associated conditioning circuitry while the distal signal is acquired using a plethysmographic sensor and associated conditioning circuitry.

The pulse wave detection system can alternatively be comprised of one sensor for recording the pulse wave. Pulse wave velocity can be estimated algorithmically using the signal from a single plethysmographic sensor. The shape of the plethysmogram wave form can be used to detect pulse wave velocity. This may be done with an empirical algorithm, for example using regression or machine learning.

In an alternative embodiment, the single plethysmographic sensor has multiple LEDs of different wavelengths. The pulse wave velocity can be estimated empirically based on the signals generated from the photodetector when excited by the different LEDs.

In another implementation, the wave form of the plethysmogram is used to empirically estimate transmural pressure directly, for example using regression or machine learning. The blood pressure can be derived from the transmural pressure with the relative external pressure.

In all implementations, the photoplethysmography sensor(s) can be placed at various locations. For optimal signal quality, the measurement site should be at a location with an artery near the surface. For the primary implementation, it is expected that the measurement site be at the finger or the wrist, but it could potentially be on another appendage.

FIG. 2 shows an embodiment of the pulse wave detection system 200 using photoplethysmography, example data acquired from these sensors, and the application of these signals in pulse wave velocity acquisition. Two photoplethysmography sensors, 210 and 211 are shown. Each photoplethysmography sensor has a light source 205, such as an LED 205, and a photodetector 204. The photodetector may be a photodiode or a photo transistor, for example. Soft tissue 203 is shown under which is an artery 201. Equations S1-S7 below show the derivation for Equation 1. As mentioned above, embodiments can employ only one photoplethysmographic sensor for a distal signal. The proximal signal may be provided using by an electrocardiogram or some other heartbeat detection device such as an ultrasound sensor to detect heart cycles or an accelerometer to detect heart cycles.

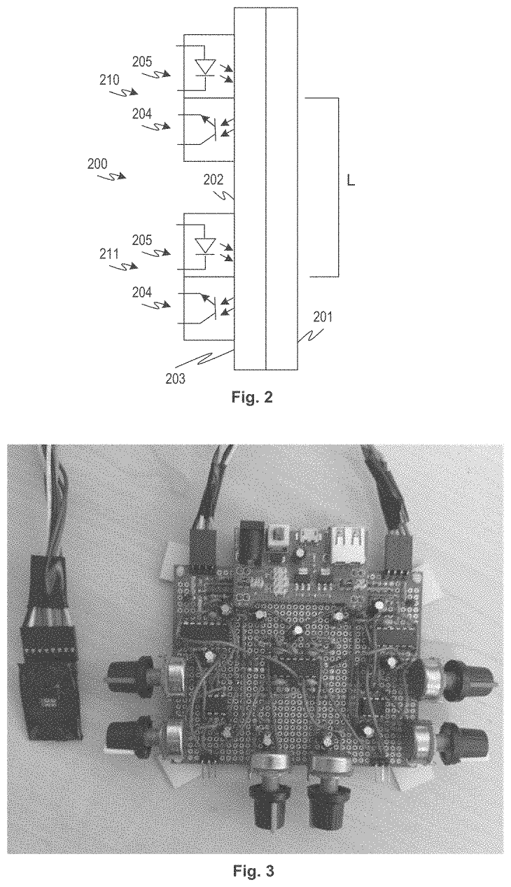

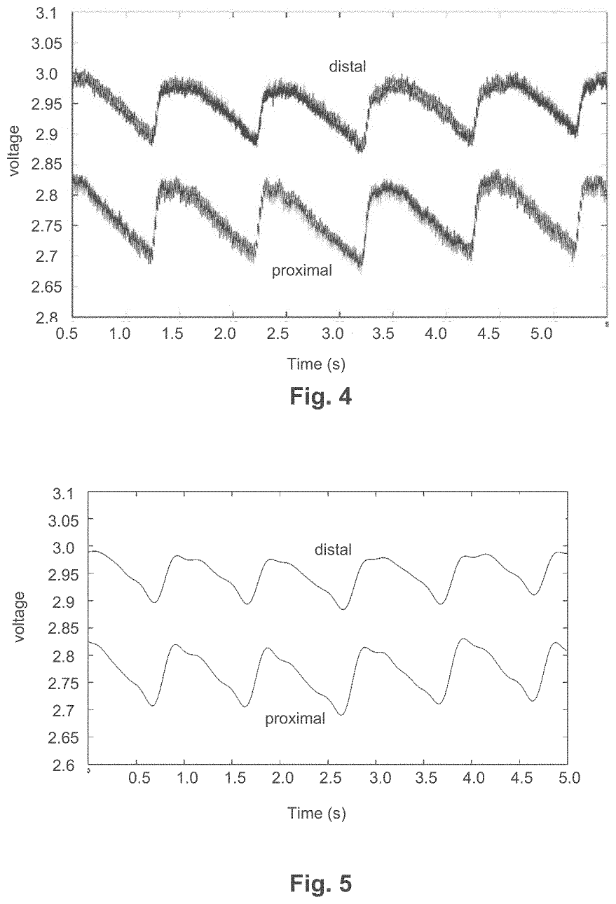

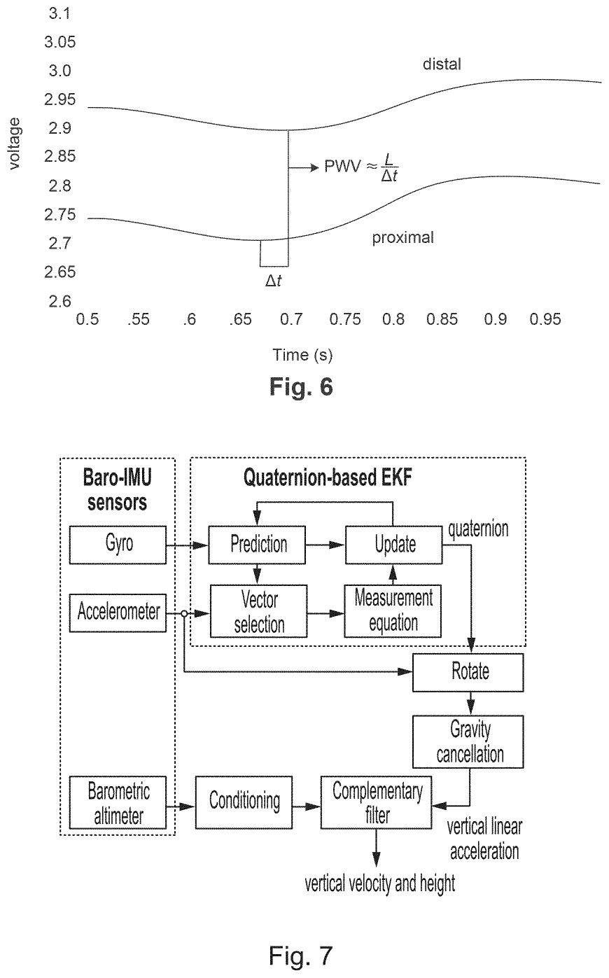

FIG. 3 shows an image of an embodiment of the photoplethysmographic device illustrated in FIG. 2. shows a photo of an embodiment of the device of FIG. 2 applied to the finger. FIG. 4 shows example data acquired from the embodiment of FIGS. 2 and 3. FIG. 5 shows an example of the data of FIG. 4 after low pass filtering. FIG. 6 illustrates how pulse wave velocity is calculated from the data of FIG. 5.

The device may further include an external pressure compensation component. External pressure compensation to account for external pressure applied to the arteries which affects the relationship between pulse wave velocity and blood pressure. To accurately estimate blood pressure based on pulse wave velocity, compensation of the external pressure may be used advantageously.

Some embodiments of the external pressure compensation component includes a pressure sensor, for monitoring the contact pressure of the device when applied to the user, and a hydrostatic pressure compensator. The hydrostatic pressure compensator may include an accelerometer, gyroscope, and barometer. In some embodiments, the signals from these three sensors are combined using an advanced sensor fusion algorithm that enables tracking altitude changes in real-time with greater accuracy and resolution than possible with the individual sensors alone. The change in altitude relative to the user's heart is used to calculate the hydrostatic pressure contribution in some embodiments by Eqn. 2.

Pₕ=μgh (2)

In Eqn. 2, ρ is the density of the blood, g is the gravitational constant, and h is the altitude relative to the heart. Tracking of hydrostatic pressure is relevant because a difference in elevation of 5 cm between the measurement site and the heart can contribute an error of 3.68 mmHg, more than 50% of the 5 mmHg error allowed by the AAMI measurement standard. Together, the pressure sensor and hydrostatic pressure compensator enable monitoring of the external pressure applied to the arteries and enable more accurate blood pressure measurement.

In an alternative implementation, the external pressure compensation component includes a muscle activation sensor in addition to a contact pressure sensor and hydrostatic pressure compensator. The muscle activation sensor is used to monitor the external pressure applied to the arteries due to muscle contraction.

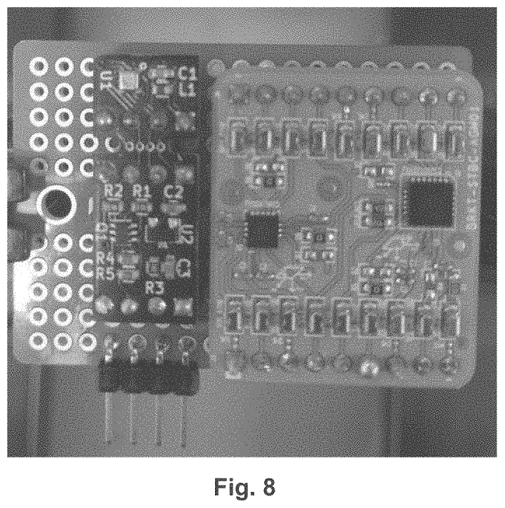

FIG. 7 shows a block diagram of the sensor fusion algorithm used for altitude tracking. FIG. 8 shows an example implementation of the hydrostatic pressure compensator. The example may have a pair of photoplethysmographic sensors, indicates how altitude tracking is performed, and gives data demonstrating the sensor fusion technique.

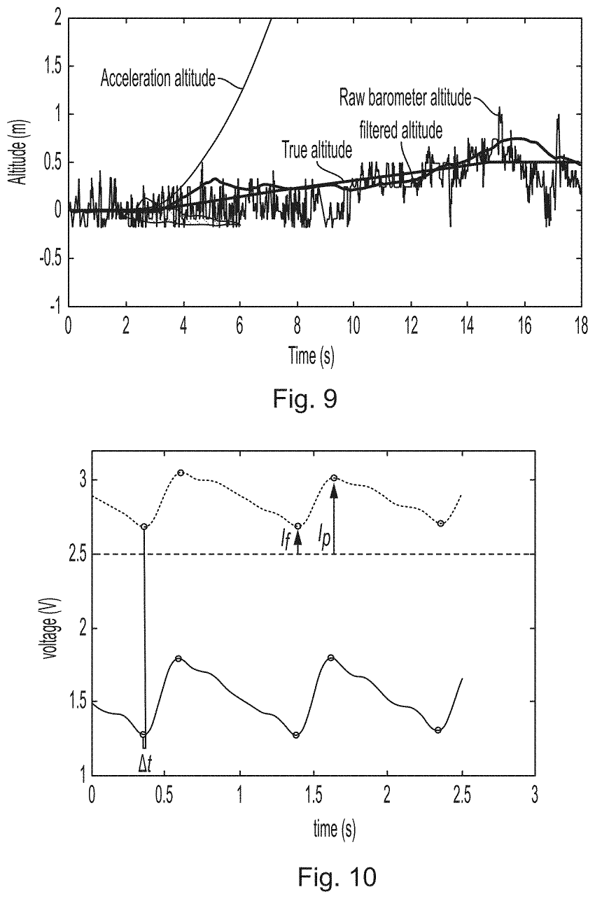

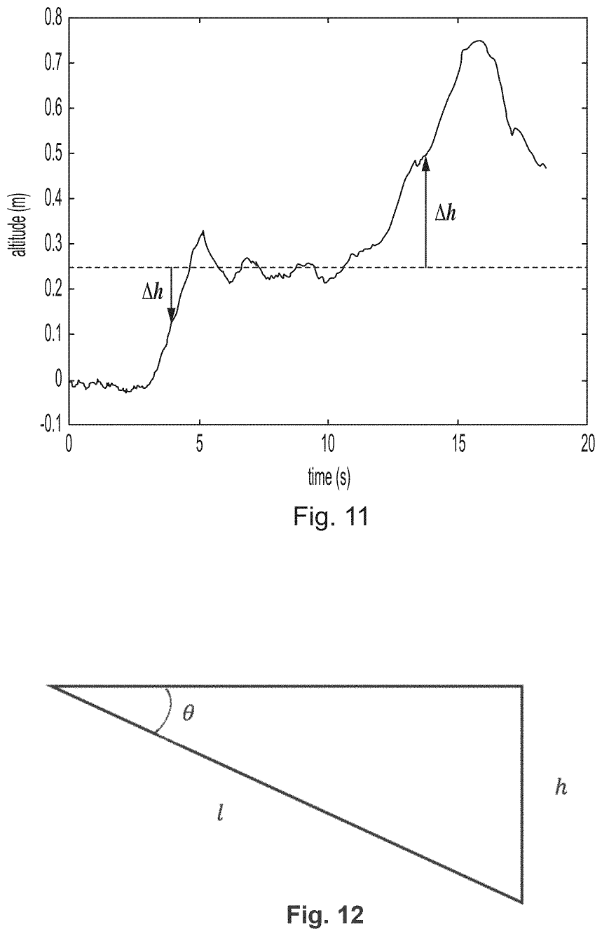

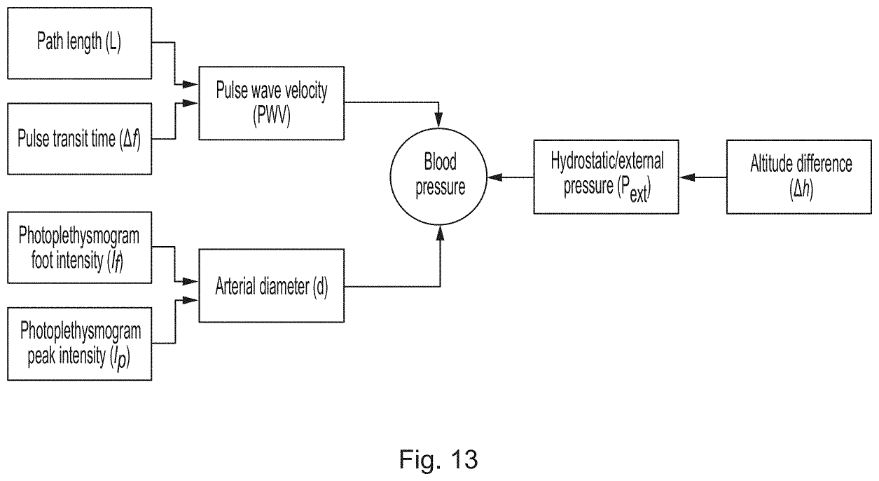

FIG. 9 shows data demonstrating the effectiveness of the sensor fusion technique. FIG. 10 shows a filtered photoplethysmography signal and parameterized features. FIG. 11 shows filtered altitude and parameterized features. FIG. 12 illustrates how relative altitude at any point can be related to path length traveled as shown in Eqn. 17. FIG. 13 shows a block diagram demonstrating use of parameterized features in blood pressure estimation.

Embodiments also includes a processing unit that utilizes the data from these sensors to algorithmically track the user's beat-to-beat blood pressure. The sensor data can be related to blood pressure through a number of techniques including, but not limited to, (1) analytical models, (2) linear regression, (3) polynomial regression, (4) machine learning, or (5) a combination of methods.

FIGS. 10, 11, and 13 show examples of the data acquired from the sensors and some of the parameterized features that may be incorporated into the blood pressure tracking algorithm. Derivation S2 (Eqns. S8-S16) is an example of how these features can be utilized to estimate blood pressure. This is not an exhaustive list of features, as some may be found later that prove predictive of blood pressure. Further, Eqn. S16 makes use of only a subset of the identified features. This limitation is because most of the identified features cannot yet be analytically related to blood pressure. As such, this is only a potential method in which these features can be utilized. The optimal algorithm(s) may be optimized for the application.

Embodiments may further include a processing unit that is used for internally calibrating the device to improve the accuracy of the blood pressure estimate. The device is calibrated by monitoring the change in external pressure over a short period of time and the effect on the signals acquired by the different sensors. By assuming blood pressure is constant over that period, the processing unit can calculate the parameters needed to fit or update the algorithm used for beat-to-beat blood pressure tracking.

In one implementation, the amplitude of the plethysmogram waveform is monitored during a period of changing external pressure and is used to calibrate the device. In another implementation, the transit time between different characteristic points in the proximal and distal pulse wave signal is monitored to calibrate the device. In another implementation, a combination of features from the sensors is used to determine the parameters needed to calibrate the device by utilizing a blood pressure tracking algorithm with no bias term.

Derivations S3-S5 demonstrate how each of these implementations may be used to internally-calibrate the device. However, the exact method for internal calibration is dependent on the algorithm that is used for tracking blood pressure. Therefore, this information is offered primarily as a conceptual example.

The calibration procedure may potentially be performed with or without user interaction. In one implementation, the calibration procedure is automatically performed when the device detects a period of changing external pressure and the conditions for assuming constant blood pressure are met. The change in external pressure can be due to changes in contact pressure, hydrostatic pressure, muscle contraction or a combination of these.

In another implementation, the calibration procedure would be performed with user assistance. When the device detects that the conditions for assuming constant blood pressure are met, the user may choose to calibrate the device. The user will then be instructed to perform a series of procedures to perturb the external pressure and thus allow the device to calibrate. For example, if the device is applied to the user's wrist, they may be instructed to slowly raise and lower their arm to alter hydrostatic pressure.

In another implementation, both forms of calibration are used. In this implementation, the automatic method may be the primary means of calibration. However, when a pre-determined period of time has passed since the last user-assisted calibration or a test for calibration quality is not passed, the user may be alerted and instructed to perform a user-assisted calibration procedure.

The embodiments may further include a processing unit that is used to detect periods of constant blood pressure to be used by the internal calibration algorithm. This processing unit monitors the signals from the various sensors and estimates when blood pressure is remaining relatively constant within a pre-determined error bound. To accomplish this, various techniques can be used including, but not limited to, (1) logistic regression classification, (2) support vector machines, (3) neural networks, (4) other machine learning classifiers, or (5) a combination of methods.

Embodiments uses real-time altitude and contact pressure tracking to monitor external pressure and compensate pulse wave velocity-derived blood pressure estimates. The device uses an internal calibration scheme for calibrating a cuffless blood pressure estimation algorithm in some embodiments. Additionally, the device can detect periods of stable blood pressure.

Embodiments may be used for ambulatory monitoring of blood pressure for patients at risk for or previously diagnosed with hypertension. Embodiments may be used by patients to monitor their response to antihypertensive drugs. Embodiments could also be used by patients prescribed medication with known blood pressure side-effects to monitor their response. Embodiments could also be used in clinical settings for the continuous, non-invasive monitoring of blood pressure of admitted patients. Embodiments could foreseeably be used as a next-generation fitness tracker that provides continuous blood pressure readings. Embodiments may also be integrated with a larger platform for Smart and Connected Health. This class of devices may be used for improved diagnosis and monitoring of hypertension.

Embodiments may provide user-friendly devices that can track meaningful health data that are accurate, minimally invasive, and unobtrusive. Blood pressure (BP) monitoring provides deep insights into a patient's health for a variety of conditions, including infectious and chronic diseases. Techniques for cuffless BP tracking based on pulse wave velocity (PWV), the velocity of the BP wave, are especially promising. However, conventional efforts rely on incomplete mathematical models and require repeated per-person calibration, inhibiting adoption. In some embodiments disclosed these models may be updated using an algorithm shown for accurate, calibration-free monitoring of BP using methods that include, for example, machine learning. Accordingly, embodiments may enable a novel class of calibration-free, continuous BP measurements devices and greatly expand the predictive power of smart and connected health.

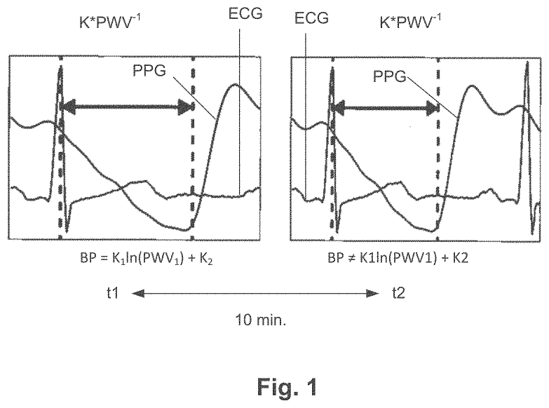

Cuffless BP monitoring from PWV has two fundamental approaches, but they both suffer due to dependence on inadequate models. The first approach estimates BP directly from these simplified models, like the one shown in FIG. 1 where K₁ and K₂ are subject-specific parameters determined through calibration. However, these coefficients are not invariant with time and must be frequently reacquired to maintain tolerable accuracy; thus, devices performing direct estimation using these models fail to accurately track BP as soon as 10 minutes after calibration, as illustrated by FIG. 1. The second approach involves the use of machine learning routines.

Because selection of features optimal for learning is especially challenging, these overly simple models have been used to guide feature selection. Subsequently, the feature vectors have been comprised of characteristics from signals used for PWV acquisition, specifically electrocardiography (ECG) and photoplethysmography (PPG). Despite the use of increasingly sophisticated learning routines, an accurate, calibration-free algorithm has been elusive, indicating that these signals may be insufficient for accurate BP tracking. Since the development of these simple models, covariates that affect the relationship between BP and PWV, like sensor contact pressure and activity, have been identified and studied. It is believed that accurate and calibration-free BP estimation necessitates tracking PWV and these covariates. Thus, machine learning may be used to develop a calibration-free algorithm for BP monitoring with feature selection guided by an updated model of BP that tracks PWV and key covariates.

We derive an updated model of BP by substituting the latest theoretical and empirical expressions into the equations for conservation of mass and momentum to develop an updated model for BP that is dependent on PWV and relevant covariates. We focus on including covariates that can be tracked using current sensors and have been demonstrated to significantly affect the dependence of BP on PWV. The effects of heartrate, hydrostatic pressure, sensor contact pressure, activity, and ambient temperature may be recorded. This expanded model may be used to inform the selection of features to provide sufficient coverage for accurate estimation of BP. Because the expanded model may account for known confounding variables, it may fit experimental data found in databases like MIMIC II better than the conventional physical models. However, because parameters like arterial dimensions cannot be easily tracked, some applications of the model may still depend on patient-specific calibration.

To acquire the data for machine learning, an integrated measurement device uses consumer sensors that can collect signals tracked by the updated physical model. An integrated prototype is preferred because it may ensure the data is consistently acquired and may minimize error attributed to timestamp mismatch. While off-the-shelf components may be used to minimize development difficulties, embodiments of the device employ a combination of sensors. For example, the device may be comprised of two PPG sensors, for acquisition of PWV in addition to the sensors necessary for covariate tracking, including: a pressure sensor, a 9 degree of freedom sensor, and a temperature sensor. The reference BP may be collected with an FDA approved continuous cuff-based device while the prototype device would concurrently collect the signals of interest. When the updated physical model is applied to this data, it may track BP accurately for a longer period than current equations after initial calibration. In some embodiments, instead of two PPG sensors, a proximal signal may be provided from ECG allowing only a single PPG to be used for the distal measurement.

After collecting data from a patient cohort using the device, the identified features may be extracted and divided into learning, validation, and testing sets. Different learning routines may be applied to the training set to generate algorithms for calibration-free estimation of BP. The resulting algorithms can be evaluated using a k-fold Monte Carlo cross-validation scheme to determine which equation performs optimally. Finally, the optimal algorithm to the testing set can be applied. The resulting BP estimates may be statistically compared to the measurements acquired with the commercial monitor to determine the accuracy of the algorithm to unseen data. This algorithm, in addition to being more accurate than conventional equations, may eliminate or significantly reduce the need for per-person calibration.

Because it is a complex trait, it may not yet be feasible to track BP completely without calibration. Therefore, if the updated calibration-free equation fails to track BP accurately, embodiments can implement an updated algorithm to allow for one-time or once-a-day calibration. Alternatively, classification routines may be used to develop a calibration-free algorithm for detecting hypo- or hypertensive events.

While algorithms for tracking BP from PWV have been developed, they fail to account for important covariates, thus limiting their accuracy and necessitating frequent recalibration.

Development and validation of the updated BP estimation algorithm enable cuffless measurement of BP and mark the next step towards the realization of smart and connected health. Further, such an advancement may be used to improve the diagnosis of hypertension and other cardiovascular diseases, diseases which are the leading cause of death in the world and contribute to an economic loss of approximately $250 billion each year in the United States alone. The research may be applied to activities and lesson plans appropriate for various outreach programs, such as Girls' Science Day.

FIG. 2 shows a design for a pulse wave detection system using photoplethysmography sensors FIG. 3 is an image of a sensor implementation with conditioning circuitry FIG. 4 shows example data acquired from this implementation of the device applied to the finger. FIG. 5 shows example of filtered data. FIG. 6 illustrates pulse wave velocity calculation.

FIG. 8 is an image of a hydrostatic pressure compensator implementation. FIG. 7 is a block diagram describing the sensor fusion algorithm used for altitude tracking (Sabatini & Genovese, 2014). FIG. 9 shows preliminary data demonstrating the effectiveness of the sensor fusion technique.

### Derivation S1: Derivation of Common Analytical Blood Pressure Tracking Algorithm

Start with the Moens-Korteweg equation describing pulse wave velocity (PWV) in terms of the elastic modulus of the artery (E), the thickness of the artery (h), blood density (ρ), and the diameter of the artery (d).

$$P W V = \sqrt{\frac{E h}{ρ d}} \qquad ( S1 )$$

Model E as a function of pressure (Pₜᵣₐₙₛ), the elastic modulus at 0 pressure (E₀), and a calibration coefficient (α). Assume that Pₜᵣₐₙₛ is equal to blood pressure (BP)

E=E₀e^{α·P}^{ₜᵣₐₙₛ}=E₀e^{α·BP} (S2)

Substitute equation S2 into equation S1 and rearrange to solve for BP

$$\begin{matrix}P W V = \sqrt{\frac{{E}_{0} h {e}^{α \cdot BP}}{ρ d}} = {e}^{0.5 α \cdot BP} \times \sqrt{\frac{{E}_{0} h}{ρ d}} & ( S3 ) \\ ln ( P W V ) = ln ( {e}^{0.5 α \cdot BP} \times \sqrt{\frac{{E}_{0} h}{ρ d}} ) & ( S4 ) \\ ln ( P W V ) = \frac{1}{2} α \cdot BP + ln ( \sqrt{\frac{{E}_{0} h}{ρ d}} ) & ( S5 ) \\ BP = \frac{2}{α} ln ( P W V ) - \frac{2}{α} ln ( \sqrt{\frac{{E}_{0} h}{ρ d}} ) & ( S6 )\end{matrix}$$

Assume that α and the ratio

$$\frac{{E}_{0} h}{ρ d}$$

are constant. Redefine equation S6 in terms of calibration coefficients K₁ and K₂

BP=K₁ ln(PWV)+K₂ (S7)

Derivation S2: Potential Blood Pressure Tracking Algorithm with Additional Features

Start with the Moens-Korteweg equation describing pulse wave velocity (PWV) in terms of the elastic modulus of the artery (E), the thickness of the artery (h), blood density (ρ), and the diameter of the artery (d).

$$P W V = \sqrt{\frac{E h}{ρ d}} \qquad ( S8 )$$

Model E as a function of pressure (Pₜᵣₐₙₛ), the elastic modulus at 0 pressure (E₀), and a calibration coefficient (α).

E=E₀e^{α·P}^{ₜᵣₐₙₛ} (S9)

Define Pₜᵣₐₙₛ as a function of blood pressure (BP) and external pressure (Pₑₓₜ)

Pₜᵣₐₙₛ=BP−Pₑₓₜ (S10)

Substitute equation S10 into equation S9

E=E₀e^{α·BP-α·P}^{ₑₓₜ}=E₀e^{α·BP}e^{-α·P}^{ₑₓₜ} (S11)

Substitute equation S11 into equation S8. Rearrange to solve for BP

$$\begin{matrix}P W V = \sqrt{\frac{{E}_{0} h {e}^{α \cdot BP} {e}^{- a P} e x t}{ρ d}} = \frac{{e}^{0.5 α \cdot BP} {e}^{- 0.5 α \cdot {P}_{e x t}}}{{d}^{0.5}} \times \sqrt{\frac{{E}_{0} h}{ρ}} & ( S12 ) \\ ln ( P W V ) = ln ( \frac{{e}^{0.5 α \cdot BP} {e}^{- 0.5 α \cdot {P}_{e x t}}}{{d}^{0.5}} \times \sqrt{\frac{{E}_{0} h}{ρ}} ) & ( S13 ) \\ ln ( P W V ) = \frac{1}{2} α \cdot BP - \frac{1}{2} α \cdot {P}_{e x t} - \frac{1}{2} ln ( d ) + ln ( \sqrt{\frac{{E}_{0} h}{ρ}} ) & ( S14 ) \\ BP = \frac{2}{α} ln ( P W V ) + {P}_{e x t} + \frac{d}{α} - \frac{2}{α} ln ( \sqrt{\frac{{E}_{0} h}{ρ}} ) & ( S15 )\end{matrix}$$

Assume that α and the ratio

$$\frac{{E}_{0} h}{ρ}$$

are constant. Redefine equation S6 in terms of calibration coefficients K₁ and K₂

$$B P = {K}_{1} ( ln ( P W V ) + \frac{d}{2} ) + {P}_{e x t} + {K}_{2} \qquad ( S16 )$$

### Derivation S3: Calibration by Monitoring Amplitude of Plethysmogram Waveform

Let mean blood pressure (MBP) be equal to the external pressure (Pₑₓₜ) that maximizes the amplitude of the plethysmogram waveform (A(PG))

$$M B P = arg {max}_{{P}_{e x t}} A ( P G ) \qquad ( S17 )$$

Let MBP be a function of pulse wave velocity (PWV), Pₑₓₜ, and calibration coefficients (K₁ and K₂). Solve for specific values of PWV and Pₑₓₜ

MBP=K₁ ln(PWV₁)+K₂+P_{ext,1} (S18)

Solve for K₁

$${K}_{1} = \frac{MBP - {P}_{ext , 1} - {K}_{2}}{ln ( {PWV}_{1} )} \qquad ( S19 )$$

Perturb Pₑₓₜ and measure PWV response. Substitute values into equation S18

MBP=K₁ ln(PWV₂)+K₂+P_{ext,2} (S20)

Substitute equation S19 into equation S20

$$MBP = \frac{MBP - {P}_{ext , 1} - {K}_{2}}{ln ( {PWV}_{1} )} ln ( {PWV}_{2} ) + {K}_{2} + {P}_{ext , 2} \qquad ( S21 )$$

Solve for K₂

$${K}_{2} = \frac{MBP - {P}_{ext , 2} - \frac{( MBP - {P}_{ext , 1} ) ln ( {PMV}_{2} )}{ln ( {PMV}_{1} )}}{1 - \frac{ln ( {PMV}_{2} )}{ln ( {PMV}_{1} )}} \qquad ( S22 )$$

Substitute equation S22 into equation S19 and solve for K₁

$${K}_{1} = \frac{MBP - {P}_{ext , 1}}{ln ( {PMV}_{1} )} - \frac{MBP - {P}_{ext , 2} - \frac{( MBP - {P}_{ext , 1} ) ln ( {PMV}_{2} )}{ln ( {PWV}_{1} )}}{( 1 - \frac{ln ( {PMV}_{2} )}{ln ( {PMV}_{1} )} ) ln ( {PWV}_{1} )} \qquad ( S23 )$$

### Derivation S4: Calibration by Monitoring Timing of Plethysmogram Waveform

Let diastolic blood pressure (DBP) be equal to the external pressure (Pₑₓₜ) that maximizes the foot-measured pulse transit time (PTT_{ƒ})

$$DBP = arg {max}_{{P}_{e x t}} {PTT}_{f} \qquad ( S24 )$$

Let systolic blood pressure (SBP) be equal to Pₑₓₜ that maximizes the peak-measured pulse transit time (PTTₚ)

$$SBP = arg {max}_{{P}_{e x t}} {PTT}_{p} \qquad ( S25 )$$

Let DBP be a function of pulse wave velocity (PWV), Pₑₓₜ, and calibration coefficients (K₁ and K₂). Solve for specific values of PWV and Pₑₓₜ

DBP=K₁ ln(PWV₁)+K₂+P_{ext,1} (S26)

Solve for K₁

$${K}_{1} = \frac{DBP - {P}_{ext , 1} - {K}_{2}}{ln ( {PWV}_{1} )} \qquad ( S27 )$$

Perturb Pₑₓₜ and measure PWV response. Use values to solve for K₂

$${K}_{2} = \frac{DBP - {P}_{ext , 2} - \frac{( DBP - {P}_{ext , 1} ) ln ( {PMV}_{2} )}{ln ( {PMV}_{1} )}}{1 - \frac{ln ( {PWV}_{2} )}{ln ( {PMV}_{1} )}} \qquad ( S28 )$$

Substitute equation 5 into equation 4 to solve for K₁

$${K}_{1} = \frac{DBP - {P}_{ext , 1}}{ln ( {PMV}_{1} )} - \frac{DBP - {P}_{ext , 2} - \frac{( DBP - {P}_{ext , 1} ) ln ( {PMV}_{2} )}{ln ( {PMV}_{1} )}}{( 1 - \frac{ln ( {PWV}_{2} )}{ln ( {PMV}_{1} )} ) ln ( {PMV}_{1} )} \qquad ( S29 )$$

Repeat the procedure to calibrate for SBP

$$\begin{matrix}SBP = {K}_{3} ln ( {PWV}_{1} ) + {K}_{4} + {P}_{e x t , 1} & ( S30 ) \\ {K}_{3} = \frac{SBP - {P}_{ext , 1} - {K}_{4}}{ln ( {PWV}_{1} )} & ( S31 ) \\ {K}_{4} = \frac{SBP - {P}_{ext , 2} - \frac{( SBP - {P}_{ext , 1} ) ln ( {PWV}_{2} )}{ln ( {PWV}_{1} )}}{1 - \frac{ln ( {PWV}_{2} )}{ln ( {PWV}_{1} )}} & ( S32 ) \\ {K}_{3} = \frac{SBP - {P}_{ext , 1}}{ln ( {PWV}_{1} )} - \frac{SBP - {P}_{ext , 2} - \frac{( SBP - {P}_{ext , 1} ) ln ( {PWV}_{2} )}{ln ( {PWV}_{1} )}}{( 1 - \frac{ln ( {PWV}_{2} )}{ln ( {PMV}_{1} )} ) ln ( {PMV}_{1} )} & ( S33 )\end{matrix}$$

Derivation S5: Calibration from Unbiased Equation

Let diastolic blood pressure (DBP) be described by a function of pulse wave velocity (PWV), external pressure (Pₑₓₜ), and calibration coefficients (K₁ and K₂) that does not have a bias term.

$$DBP = {K}_{1} {e}^{- \frac{{K}_{2}}{{PWV}_{1}}} + {P}_{e x t} \qquad ( S34 )$$

Perturb Pₑₓₜ and measure the effect on PWV. Repeat for three different measurements.

Simultaneously solve the three equations to find current DBP and calibration coefficients.

$$\begin{matrix}DBP = {K}_{1} {e}^{- \frac{{K}_{2}}{{PWV}_{1}}} + {P}_{{ext}_{1}} & ( S35 ) \\ DBP = {K}_{1} {e}^{- \frac{{K}_{2}}{{PWV}_{2}}} + {P}_{{ext}_{2}} & ( S36 ) \\ DBP = {K}_{1} {e}^{- \frac{{K}_{2}}{{PWV}_{3}}} + {P}_{{ext}_{3}} & ( S37 )\end{matrix}$$

Repeat analysis with SBP to calibrate algorithm

$$\begin{matrix}SBP = {K}_{3} {e}^{- \frac{{K}_{4}}{{PWV}_{1}}} + {P}_{{ext}_{1}} & ( S38 ) \\ SBP = {K}_{3} {e}^{- \frac{{K}_{4}}{{PWV}_{2}}} + {P}_{{ext}_{2}} & ( S39 ) \\ SBP = {K}_{3} {e}^{- \frac{{K}_{4}}{{PWV}_{3}}} + {P}_{{ext}_{3}} & ( S40 )\end{matrix}$$

The embodiments have the following characteristics:

Embodiments include ones in which an external pressure compensation unit for tracking the effect of contact pressure, hydrostatic pressure, and, potentially, smooth muscle contraction. This accounts for the effects of external pressure which may negatively impact the accuracy of the BP estimate.

The disclosed subject matter includes an internal calibration scheme to update the BP tracking algorithm. This allows the device to be calibrated for improved accuracy for individual users which is distinct from external measurement for a one-point calibration.

The disclosed subject matter includes embodiments which contains a mechanism for detecting when BP is stable to allow for internal calibration. This predicts when BP is stable.

The disclosed subject matter includes embodiments in which two photoplethysmography sensors are used and separated by a known distance to estimate local pulse wave velocity. Other embodiments may use ECG as the proximal signal and photoplethysmography as the distal signal to calculate pulse transit time. In these cases, the path length may be inferred to apply the algorithm.

The disclosed subject matter includes embodiments in which one plethysmography sensor is used to estimate pulse wave velocity, transmural pressure, or blood pressure.

The disclosed subject matter includes an external pressure compensation unit to account for the effects of external pressure.

The disclosed subject matter includes embodiments with an internal calibration scheme.

The disclosed subject matter includes embodiments that detect when BP is stable for internal calibration.

### Example Methods and Results

Some embodiments disclosed provide a process for correcting blood pressure estimates generated from cuffless blood pressure monitors to compensate for error due to the external pressure at the measurement site. Some sources of external pressure considered are hydrostatic pressure, contact pressure, pressure from smooth muscle contraction, and pressure from vasoconstriction. However, this process provides a framework for accounting for any source of external pressure.

Embodiments of the disclosed subject matter include 3 elements:

(1) a signal acquisition element,

(2) a signal processing element, and

(3) an external pressure compensation element.
a. The signal acquisition element includes of a collection of sensors used to collect information related to external pressure.

In one implementation, the acquisition system contains an accelerometer, gyroscope, magnetometer, and barometer. The data from these sensors can be used to track the relative altitude of the measurement site compared to the user's heart, thereby enabling hydrostatic pressure compensation.

In a second implementation, the acquisition system contains a force sensitive sensor, such as a force sensitive resistor or a force sensitive capacitor, that can measure the pressure of the device when applied to the user, thus enabling contact pressure compensation.

In a third implementation, the acquisition system contains a muscle activation sensor that is used to monitor external pressure due to muscle contraction.

In a fourth implementation, the acquisition system contains a sensor for monitoring the diameter of the artery at the measurement site to allow for compensation of external pressure due to vasoconstriction.

Another implementation contains multiple sensors to enable tracking a combination of external pressure sources.

(2) The signal processing element is used to process the data from the different sensors such that they can be used to correct blood pressure estimates. In some embodiments, for hydrostatic pressure, data from the accelerometer, gyroscope, magnetometer, and barometer are combined using an advanced sensor fusion algorithm that enables tracking altitude changes in real-time with greater accuracy and resolution than possible with the individual sensors alone. The sensor fusion technique is illustrated by Eqn. 1A where relative altitude at the measurement site (h*) is given by a function of readings from the accelerometer ( [symbol] ), gyroscope ( [symbol] ), magnetometer ( [symbol] ), and barometer (p_{baro}). The notation can be simplified by absorbing the signals from these different signals into a single sensor term (sₕ), yielding Eqn. 1B.

h*=ƒ( [symbol] , [symbol] , [symbol] ,p_{baro}) (1A)

h*=ƒ(sₕ) (1B)

This relative altitude can then be used to correct for hydrostatic pressure.

For contact pressure, the signal from the force sensitive sensor is used to calculate the contact force applied to the user. This force is then converted to contact pressure by dividing by the surface area of the force sensitive sensor.

Calculation of contact pressure is illustrated in Eqn. 2A where contact pressure (p_{c}) is given by a function of the signal from the sensor (g(s_{c})) divided by the surface area (A).

$${p}_{c} = \frac{g ( {s}_{c} )}{A} \qquad ( 2 A )$$

Note that A is a constant for a given implementation. To simplify notation, absorb A into the function g to describe p_{c} only in terms the signal from the sensor (s_{c}), yielding Eqn. 3A.

p_{c}=α(s_{c}) (3A)

For muscle contraction and vasoconstriction, signals from the associated sensor are acquired and filtered to remove high frequency noise and baseline drift. Optionally, data from these sensors may be used to correct their effect on external pressure and blood pressure error using machine learning.

(3) After processing the signals, they can be used to compensate the error due to external pressure in some embodiments. To do so, first let the pressure estimated from a cuffless blood pressure monitor be the transmural pressure (pₜᵣₐₙₛ) (preferably the lone PG), the difference between arterial pressure (pₐ) (arterial pressure is the same as blood pressure) and external pressure (pₑₓₜ). The definition of transmural pressure is illustrated by Eqn. 4A.

pₜᵣₐₙₛ=pₐ−pₑₓₜ (4A)

Next, let the arterial pressure be defined as ‘blood pressure’ (p) and decompose external pressure into hydrostatic pressure, contact pressure, pressure due to muscle contraction, and pressure due to vasoconstriction, given by Eqn. 5A. [more accurately stated—measuring the change in external pressure because a baseline is used—ideally-call it Pₑₓₜ Signals are acquired by the system (1) and processed in the step 2 device in the system to estimate the change in external pressure. Ptrans using the favorite single plethysmogram; after have that the blood pressure for that cardiac cycle can be calculated]

pₜᵣₐₙₛ=p−(pₕ+p_{c}+pₘ+pᵥ) (5A)

Rearrange, solving for blood pressure, yielding Eqn. 6A.

p=pₜᵣₐₙₛ+pₕ+p_{c}+pₘ+pᵥ (6A)

The rest of the compensation technique of the embodiment depends on if cuffless monitor utilizes a path-independent or -dependent measure for estimating blood pressure.

In the case of a path-independent variable, first replace pₜᵣₐₙₛ with a function of the path-independent variable (ƒ(θ)), yielding Eqn. 7A.

p=ƒ(ø)+pₕ+p_{c}+pₘ+pᵥ (7A)

Next, calculate hydrostatic pressure using Eqn. 8A where h is the relative altitude where θ is measured.

pₕ=μgh (8A)

Then, substitute Eqn. 3A and Eqn. 8A into Eqn. 7A to yield Eqn. 9A.

p=ƒ(θ)+μgh+α(s_{c})+pₘ+pᵥ (9A)

Next, let the pressure due to muscle contraction and vasoconstriction be defined by machine learning models with the signal from their associated sensors as the independent variable. Substitute these models into Eqn. 9A to yield Eqn. 10A.

p=ƒ(θ)+μgh+α(s_{c})+β(sₘ)+γ(sᵥ) (10A)

Finally, note that h is equal to h* if θ and altitude measurement site are the same, yielding Eqn. 11A, an equation for blood pressure with external pressure compensation.

p=ƒ(θ)+μgh*+α(s_{c})+β(sₘ)+γ(sᵥ) (11A)

An example of a path-independent measure that could be used in this equation is pulse wave velocity (PWV), the velocity of the blood pressure wave as it travel through the arterial network. Substituting θ with PWV yields Eqn. 11B, a method for correcting PWV-derived blood pressure estimates.

p=ƒ(PWV)+μgh*+α(s_{c})+β(sₘ)+γ(sᵥ) (11B)

In the case of path-dependent measures, additional steps are required. First, define the path-dependent measure (ϕ) as the function of a path-independent measure integrated with respect to path (l), yielding Eqn. 12A.

ϕ=∫₀^{L}g(θ)·dl (12A)

Next, rearrange Eqn. 10A and substitute into Eqn. 12A to yield Eqn. 13A.

ϕ=∫₀^{L}g(ƒ⁻¹(p−(μgh+α(s_{c})+β(sₘ)+γ(sᵥ))))·dl (13A)

An example of a path-dependent measure that could be used in this equation is pulse arrival time (PAT), the time it takes for the blood pressure wave to travel from the heart to some distal site, commonly the finger tip. Using PAT as the path-dependent measured, Eqn. 13A can be rewritten as Eqn. 13B.

$$PAT = {\int }_{0}^{L} \frac{1}{{f}^{- 1} ( p - ( ρ g h + α ( {s}_{c} ) + β ( {s}_{m} ) + γ ( {s}_{v} ) ) )} \cdot dl \qquad ( 13 B )$$

Evaluating these integrals yield an equation for blood pressure that has been corrected for the effects of external pressure. However, the exact solution depends on the form of the different functions, thus the solutions may vary for different implementations and may be numerically calculated.

To illustrate a concrete example of how this could be accomplished analytically for PAT, assume that ƒ is a linear model parameterized by the constants K₁ and K₂. Thus, Eqn. 13B can be rewritten as Eqn. 14A.

$$PAT = {\int }_{0}^{L} \frac{{K}_{1}}{p - ( ρ g h + α ( {s}_{c} ) + β ( {s}_{m} ) + γ ( {s}_{v} ) ) - {K}_{2}} \qquad ( 14 A )$$

Further, assume that the effects due to contact pressure, muscle contraction, and vasoconstriction are negligible. Thus, Eqn. 14A reduces to Eqn. 15A.

$$PAT = {\int }_{0}^{L} \frac{{K}_{1}}{p - ρ g h - {K}_{2}} \qquad ( 15 A )$$

Simplify be defining new constants K₃, K₄, and K₅ to yield Eqn. 16A.

$$PAT = {\int }_{0}^{L} \frac{1}{{K}_{3} \cdot p + {K}_{4} h + {K}_{5}} \cdot dl \qquad ( 16 A )$$

Next note that relative altitude, h, is a function of the distance the wave has traveled (1). Thus, h must be redefined in terms of l. For illustrative purposes, assume that the signal is being measured at the finger such that the wave path is down the arm. Next assume that the path of wave travel is straight (e.g. the arm is fully extended) such that relative altitude at any point can be related to path length traveled using Eqn. 17A.

h=l·sin(θ) (17A)

This relationship is further demonstrated by FIG. 12.

If it is assumed that the angle (θ) of the path does not change during a cardiac cycle, then it can be found by substituting the length of the arm (L), and altitude at the finger (h*), yielding Eqn. 18A.

$$θ = arcsin ( \frac{{h}^{*}}{L} ) \qquad ( 18 A )$$

Now, combine Eqn. 18A and Eqn. 17A and substitute into Eqn. 16A to yield Eqn. 19A.

$$PAT = {\int }_{0}^{L} \frac{1}{{K}_{3} \cdot p + {K}_{4} \cdot \frac{{h}^{*}}{L} \cdot l + {K}_{5}} \cdot dl \qquad ( 19 A )$$

Integrate, define new constants, and rearrange to solve for p, yielding Eqn. 20A.

p=A·h*[exp(B·h*·PAT)−1]⁻¹+C (20A)

This equation can be used to calculate blood pressure using PAT while compensating for the effects of hydrostatic pressure under the given assumptions in some embodiments.

In an alternative implementation, Eqn. 13B can be recast as a machine learning problem. With machine learning, external pressure compensated blood pressure can be found through Eqn. 21A where the function ƒ is approximated using machine learning techniques and is a function of the signals from the various sensors and PAT.

p=ƒ(sₕ,s_{c},sₘ,sᵥ,PAT) (21A)

Disclosed is a general technique for correcting cuffless blood pressure estimates in real time by compensating for external pressure. A pilot study has been conducted that demonstrates that embodiments can significantly improve the accuracy of PAT-derived blood pressure estimates. As accuracy is a significant roadblock to conventional cuffless blood pressure monitors, the disclosed process may improve the utility of embodiments of the disclosed subject matter.

This disclosed subject matter may foreseeably be used as part of a cuffless blood pressure device to improve its accuracy. Further, it could be used as part of an internal calibration system for cuffless blood pressure monitors.

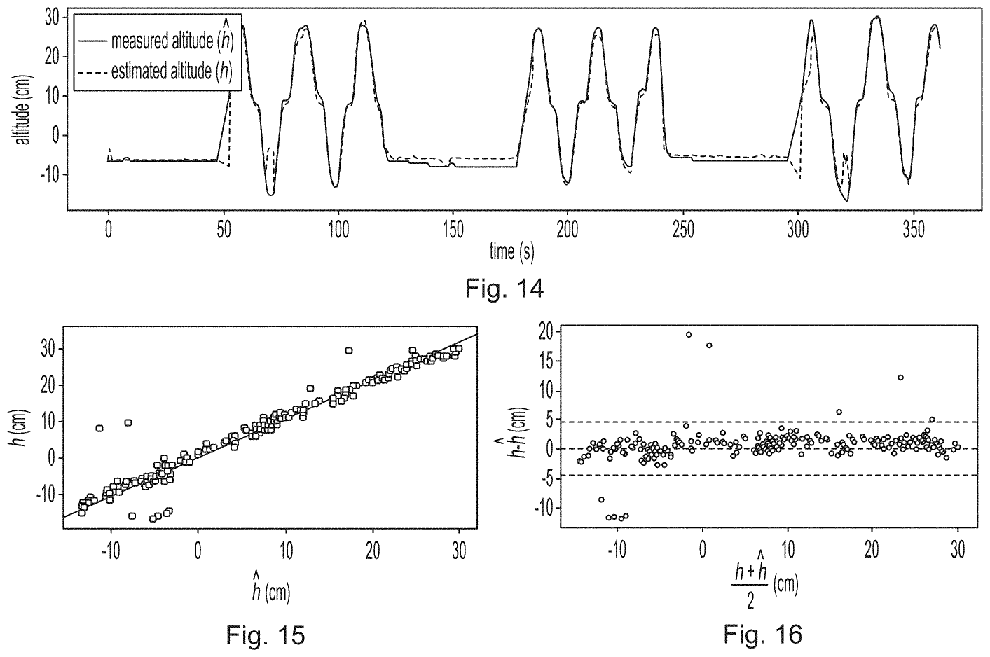

FIGS. 14-16 shows how embodiments of the external pressure compensation unit tracks hydrostatic effects. A random forest regression model was used to track relative altitude changes using the 10-degree-of-freedom sensor. The time series plot in FIG. 14 shows that these predictions closely follow the reference from the Nexfin. The correlation plot of FIG. 15 shows that the predicted and measured values have a strong correlation (R2=0.97), and the Bland-Altman plot of FIG. 16 shows that there is good agreement between these measures (MAE=1.44±1.51 cm) where the dotted line indicates 95% limits of agreement for the mean difference (grey dotted line).

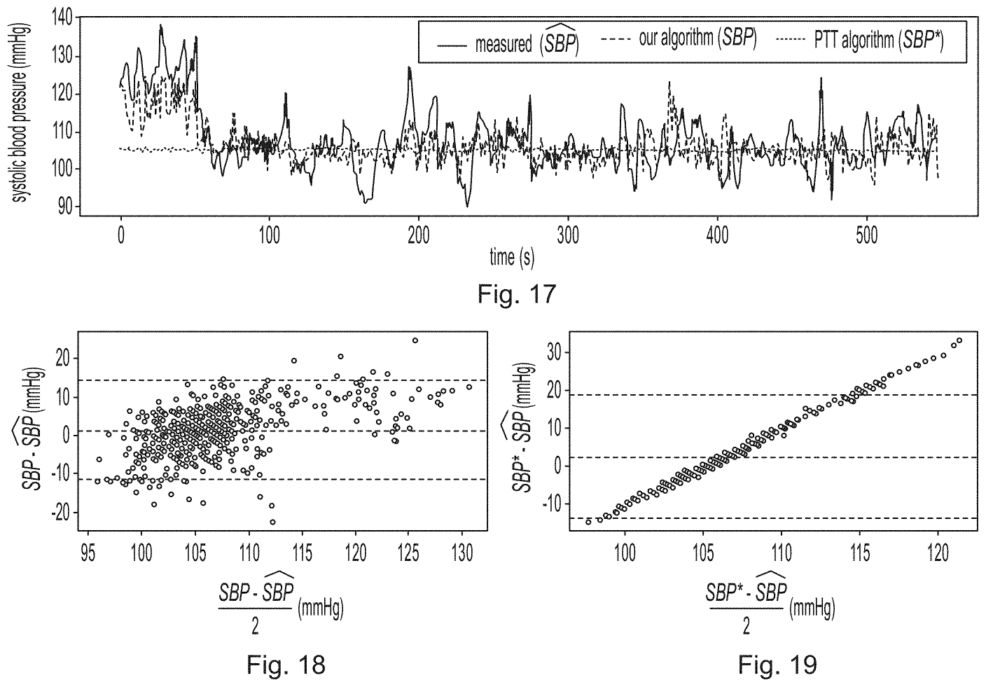

FIGS. 17-19 show how external pressure compensation improves systolic pressure estimation accuracy. A random forest regression model was used to track systolic blood pressure. The time series plot of FIG. 17 shows that the predictions using our technology tracks the reference from the Nexfin better than a competing PTT-based algorithm. The Bland-Altman plot for our algorithm (6.43±5.09 mmHg) (FIG. 18) and the PTT-based algorithm (8.95±8.69 mmHg) (FIG. 19) shows that our estimates have improved accuracy and agreement with the reference (p<0.0001).

### Section 2.1

Sections 2.1 and 2.2 disclose another set of embodiments for blood pressure monitoring. Blood pressure measurement at peripheral sites like the wrist (using current cuff-based or emerging cuffless methods) are susceptible to significant errors from hydrostatic pressure fluctuations that arise from changes in the elevation of the measurement site(s) relative to the heart. One technique to compensate for these errors is to track the relative elevation of the measurement site(s). Thus, for blood pressure measurement at the wrist, the position of the upper extremities can be tracked, specifically the upper arm and forearm. Upper arm and forearm position relative to the shoulder can be tracked by measuring the orientation of the limbs using an inertial sensor (e.g., an accelerometer). If the length of each limb is known, the position of the limbs with respect to the shoulder can be directly calculated. However, this approach requires inertial sensors on both the upper arm and forearm and thus may be difficult to adapt to a single-site wrist-wearable device.

Embodiments correct for hydrostatic pressure errors by tracking the position of the upper extremities, such as the upper arm and forearm, using wearable sensors (e.g., wearable sensors at the wrist). For example, a suite of inertial and optional time-of-flight sensors assembled in a wrist-wearable form factor can be implemented. The sensors can be used to track the orientation of the upper arm and forearm. If the length of each limb is known, this enables calculation of the position of the limbs with respect to the shoulder.

Embodiments include a 3-axis accelerometer that measures the linear acceleration of the wrist. If the forearm is at rest, these acceleration measurements can be used to calculate the pitch and roll orientation angles of the forearm. In addition to an accelerometer, embodiments can include additional optional sensors such as:
A 3-axis gyroscope, which can be used to measure the angular velocity along the sensor axes. When combined with the accelerometer, these measurements can be used to calculate yaw, pitch, and roll angle rates. By integrating these values, it is possible to calculate the change in orientation.
A 3-axis magnetometer, which can be used to measure the local magnetic field, which can be used to calculate the yaw orientation angle.
A time-of-flight sensor (e.g., radar, lidar, etc.), which can be used to measure the distance to a reference site with known position, such as the ground or the torso. Given the orientation of the forearm and known limb lengths, measurements from this sensor can be used to constrain upper arm orientation using trigonometry.

Measurements from these sensors are then used to track the orientation of the upper arm. Embodiments can achieve upper arm tracking using a non-autoregressive approach or an autoregressive approach.

Embodiments estimate the upper arm orientation directly using sensor measurements, as demonstrated in Eqn. 22:

yₜ=ƒ(x_{t-N}, . . . xₜ, . . . x_{t+M}) (22)

In Eqn. 22, y is the upper arm orientation and x are the sensor measurements. An advantage of this approach is that the cumbersome calibration procedure associated with conventional techniques is not necessary. Rather, upper arm orientation is estimated directly using the sensor data. However, a limitation of this approach is that the system is under-constrained when only inertial sensors (i.e., without a time-of-flight sensor) are used because multiple upper arm orientations could give rise to the same sensor measurements at the wrist. Therefore, occasionally large errors may be encountered. To overcome this limitation, a time-of-flight sensor can be incorporated in some embodiments. The time-of-flight sensor can be used to approximate upper arm orientation and bound the error.

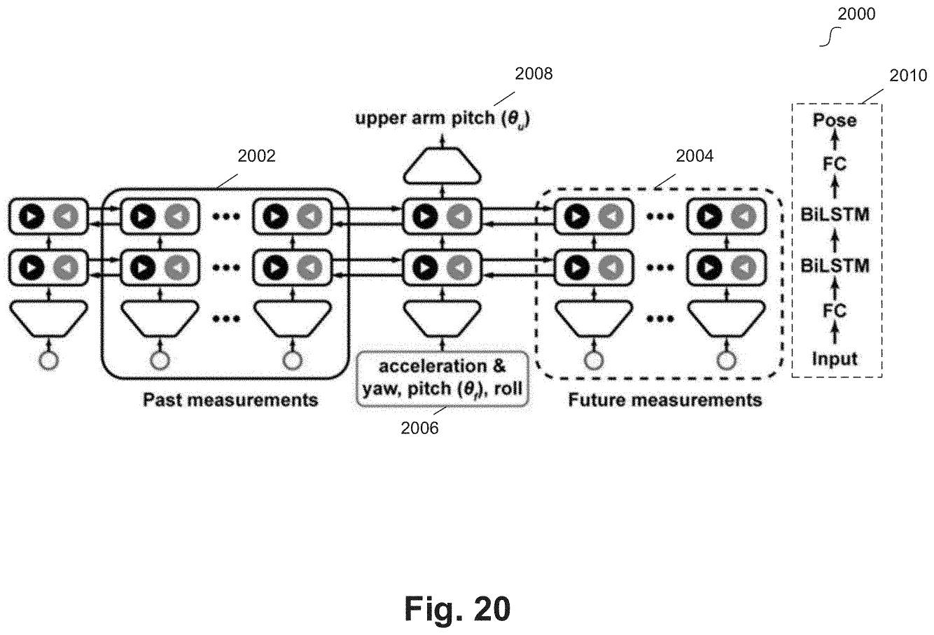

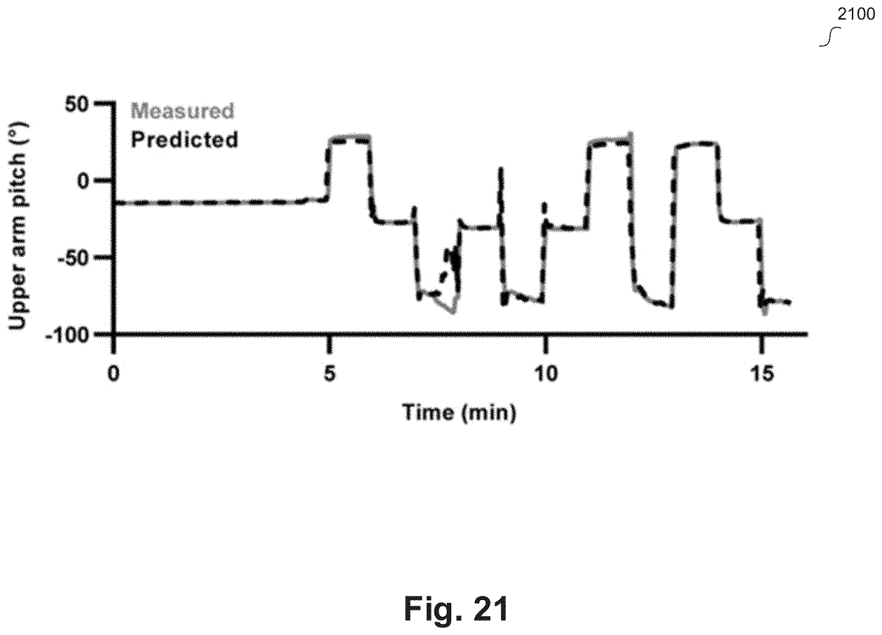

Embodiments of the non-autoregressive approach may be implemented using a deep learning model. FIG. 20 illustrates a deep learning model in accordance with some embodiments. Deep learning model 2000 can be a bi-directional long-short term memory (“LSTM”) with past measurements 2002, future measurements 2004, and current measurements 2006 used to estimate the upper arm orientation 2008. In some embodiments, layers 2010 of deep learning model 2000 can include an input layer, first fully connected layer, first bi-directional LSTM layer, second bi-directional LSTM layer, second fully connected layer, and an output layer. Any other suitable layer orientation, recurrent neural network units (e.g., gated recurrent unit (“GRU”), and the like) can be similarly implemented.

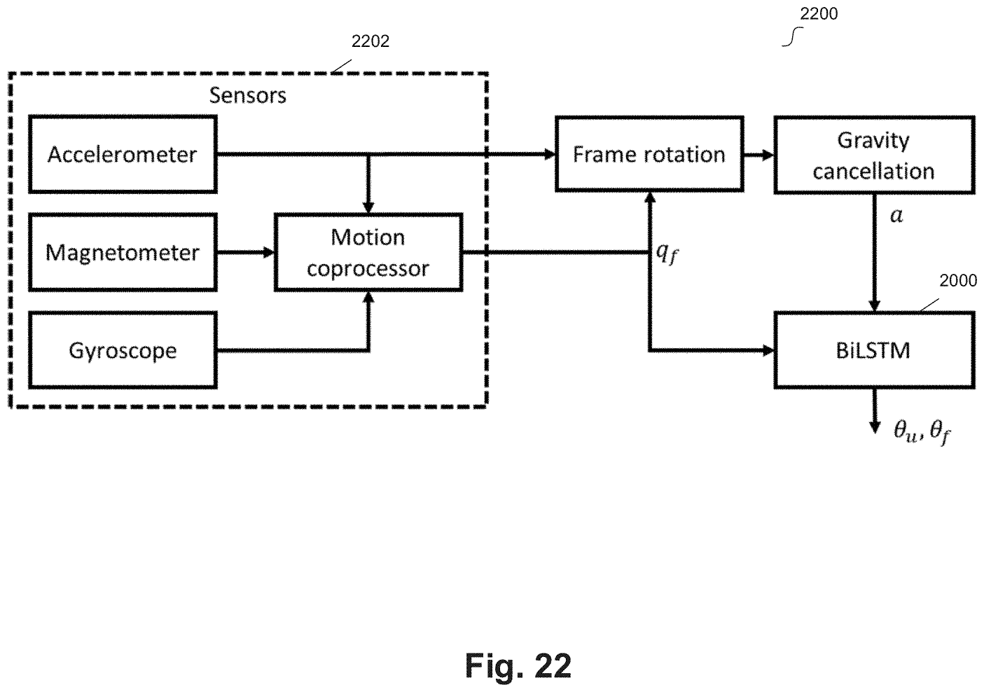

FIG. 22 illustrates a block diagram of an algorithm used to estimate arm orientation that takes a non-autoregressive approach. Sensors 2202 of algorithm 2200 include an accelerometer, gyroscope, and magnetometer. These sensors are fused using a hardware Kalman filter to measure the orientation (e.g., yaw, pitch, and roll) of the forearm in some embodiments. The forearm acceleration with gravity subtracted (a) and orientation unit quaternion (q_{ƒ}) is then fed into bidirectional LSTM architecture (e.g., as input into deep learning model 2000 of FIG. 20) to directly estimate the upper arm orientation (e.g., pitch). Across 20 human subjects, the model illustrated in FIG. 20 had a mean absolute error of 4.5±11.2°. FIG. 21 illustrates a graph of the time series of the predictions of an example subject.

In other embodiments, any other suitable deep learning architecture can be implemented. For example, suitable performance can be achieved using a convolutional neural network (e.g., composed of 1-d convolution layers), linear or bilinear recurrent neural networks with gated recurrent units and/or LSTM cells, a self-attention based transformer model, a combination of these, ensemble learning, or any other suitable architecture.

Another approach to estimating upper arm orientation taken in some embodiments is an auto-regressive approach. The task in the auto-regressive approach is to estimate the upper arm orientation using the sensor measurements given the previous upper arm orientations, as demonstrated in Eqn. 23:

yₜ=ƒ(x_{t-N}, . . . xₜ, . . . x_{t+M}|y_{t-N}, . . . yₜ₋₁) (23)

To implement the auto-regressive approach, an initial N upper arm orientations are used. To acquire these measurements, a simple calibration procedure can be performed. For example, the user could move their arm to a predetermined position (e.g., point the arm straight down along the body) for a predetermined length of time. After calibration, the known upper arm orientation and sensor measurements can be used to initialize the tracking algorithm. A limitation of this approach is that the upper arm orientation estimates could diverge from the true value over time. To overcome this limitation, intermittent recalibration can be used to ensure accuracy is maintained.

An example of this approach is an Unscented Kalman Filter that tracks the angle, angular velocity, and angular acceleration for the upper arm and forearm. For example, a system containing an accelerometer and gyroscope can measure upper arm pitch (θᵤ,), forearm pitch rate (θ_{ƒ}), and linear acceleration at the wrist (a). However, this is an under-constrained system that may not converge. To overcome this problem, a time-of-flight sensor can be included to constrain upper arm pitch (θᵤ), for example by measuring the distance between the wrist and the torso parallel to the ground (dₓ). Eqn. 24 and Eqn. 25 demonstrate a system containing these sensors:

$$\begin{matrix}x = [ \begin{matrix}{θ}_{u} \\ {\ddot{θ}}_{u} \\ {\ddot{θ}}_{u} \\ {θ}_{f} \\ {\dot{θ}}_{f} \\ {\ddot{θ}}_{f}\end{matrix} ] , f ( x ) = [ \begin{matrix}{θ}_{u} + {\dot{θ}}_{u} Δ t + {\ddot{θ}}_{u} Δ {t}^{2} \\ {\dot{θ}}_{u} + {\ddot{θ}}_{u} Δ t \\ {\ddot{θ}}_{u} \\ {θ}_{f} + {\dot{θ}}_{f} Δ t + {\ddot{θ}}_{f} Δ {t}^{2} \\ {\dot{θ}}_{f} + {\ddot{θ}}_{f} Δ t \\ {\ddot{θ}}_{f}\end{matrix} ] , & ( 24 ) \\ h ( x ) = [ \begin{matrix}{d}_{x} \\ {θ}_{f} \\ {\dot{θ}}_{f} \\ {a}_{z}\end{matrix} ] = f ( x ) = [ \begin{matrix}{L}_{u} cos {θ}_{u} + {L}_{f} cos {θ}_{f} \\ {θ}_{f} \\ {\dot{θ}}_{f} \\ {L}_{u} ( {\ddot{θ}}_{u} cos {θ}_{u} - {\dot{θ}}_{u}^{2} sin {θ}_{u} ) + {L}_{f} ( {\ddot{θ}}_{u} cos {θ}_{u} - {\dot{θ}}_{u}^{2} sin {θ}_{u} ) - g\end{matrix} ] , & ( 25 )\end{matrix}$$

In Eqn. 24 and Eqn. 25, x is the state vector, ƒ(x) is the state transition function, h(x) is the measurement function, Δt is the time between measurements, Lᵤ and L_{ƒ} are upper arm and forearm length, a_{z} is vertical acceleration, and g is acceleration due to gravity.

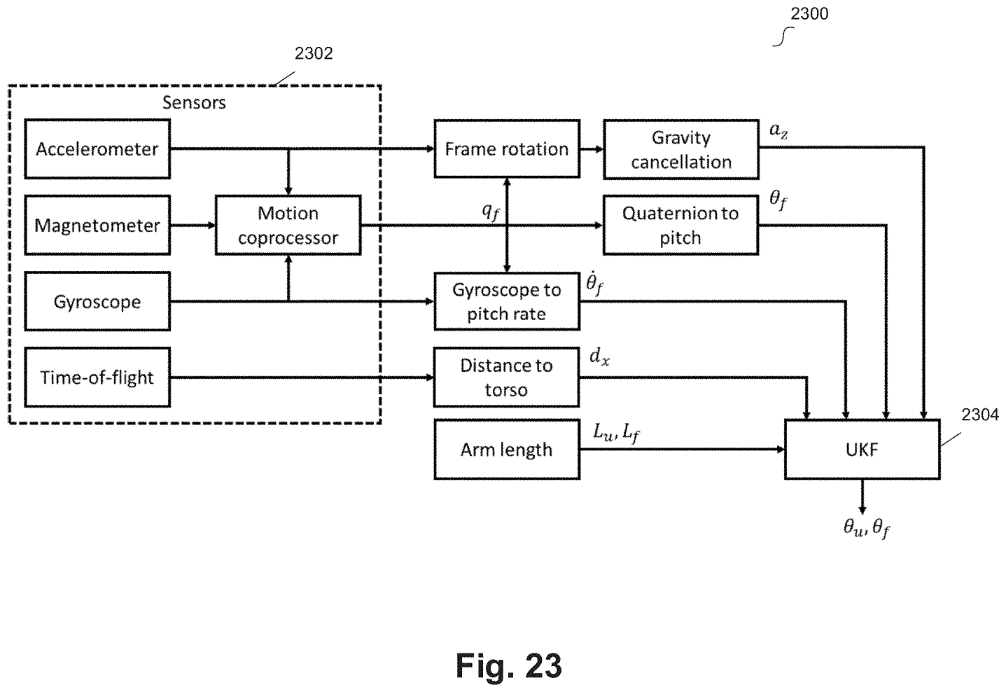

FIG. 23 illustrates a block diagram of an algorithm with a time-of-flight sensor used to estimate arm orientation that takes an autoregressive approach. Sensors 2302 of algorithm 2300 include an accelerometer, gyroscope, magnetometer, and a time-of-flight sensor. These sensors are fused using a hardware Kalman filter to measure the orientation (e.g., yaw, pitch, and roll) of the forearm in some embodiments. The forearm acceleration with gravity subtracted a_{z}, quaternion to pitch θ_{ƒ}, gyroscope to pitch rate θ_{ƒ}, distance to torso dₓ, and arm lengths Lᵤ and L_{ƒ} are then fed into Unscented Kalman Filter 2304 to directly estimate the upper arm orientation (e.g., pitch).

Another approach for estimating upper arm orientation taken in some embodiments is a hybrid approach. For example, a hybrid approach can be implemented where a non-autoregressive model is first used to estimate an unobservable parameter, such as upper arm orientation or orientation rate. Next, an autoregressive model uses this estimated parameter along with the sensor measurements to track upper arm orientation. If the non-autoregressive model estimates upper arm orientation directly, the autoregressive model can be interpreted as a type of smoothing filter.

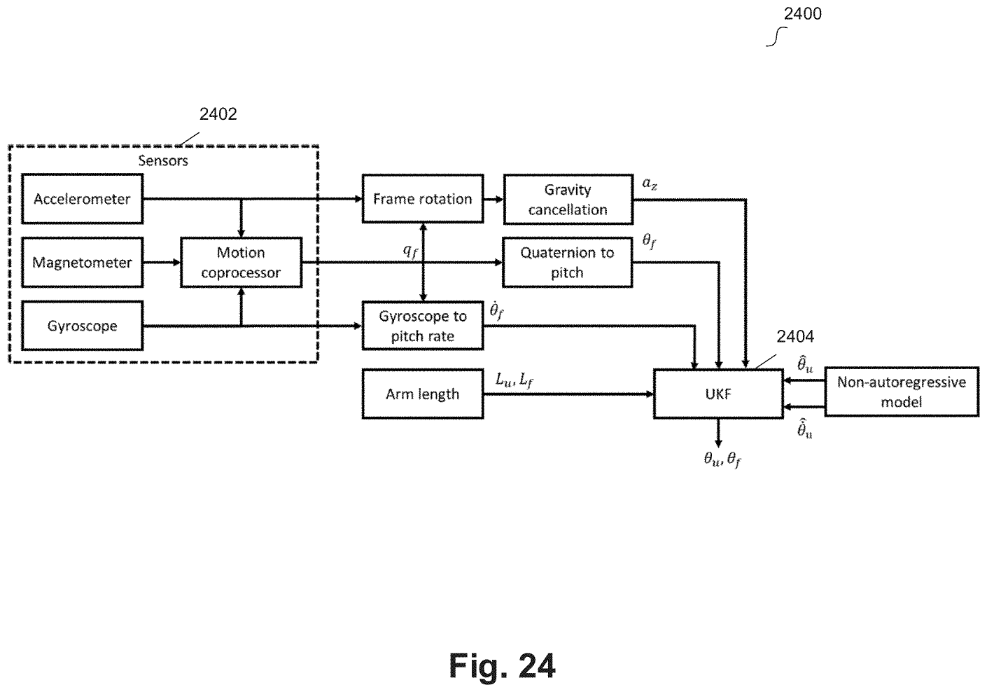

FIG. 24 illustrates a block diagram of an algorithm used to estimate arm orientation that takes a hybrid approach. Sensors 2402 of algorithm 2400 include an accelerometer, gyroscope, magnetometer, and a time-of-flight sensor. These sensors are fused using a hardware Kalman filter to measure the orientation (e.g., yaw, pitch, and roll) of the forearm in some embodiments. The forearm acceleration with gravity subtracted a_{z}, quaternion to pitch θ_{ƒ}, gyroscope to pitch rate θ′_{ƒ}, arm lengths Lᵤ and L_{ƒ}, and input from the non-autoregressive model that models unobservable parameters (e.g., upper arm orientation and/or orientation rate) are then fed into Unscented Kalman Filter 2404 to directly estimate the upper arm orientation (e.g., pitch).

In some embodiments, after calculating upper arm and forearm pitch, hydrostatic pressure errors can be corrected. For blood pressure measurement devices that measure the local blood pressure at the measurement site, such as cuff-based oscillometric monitors, hydrostatic pressure errors can be calculated in a straightforward manner as Eqn. 26:

error_{P}_{ₕ}=−pg(Lᵤ sin θᵤ+L_{ƒ} sin θ_{ƒ}) (26)

In this example, a positive pitch, θ, indicates movement of the associated limb upward. This error can be subtracted directly from the local blood pressure measurement to yield an approximation of the true central blood pressure as in Eqn. 27:

BP=BP_{wrist}−pg(Lᵤ sin θᵤ+L_{ƒ} sin θ_{ƒ}) (27)

For other approaches that estimate the central blood pressure (e.g., typically following calibration), such as those based on pulse transit time, the hydrostatic pressure correction may depend on the type of algorithm used for blood pressure prediction. For example, given blood pressure prediction using a pulse transit time algorithm, the following model represented by Eqn. 28, Eqn. 29, and Eqn. 30 can be derived starting from the Moens-Korteweg and Hughes equations:

$$\begin{matrix}P = \frac{1}{{k}_{2}} ln \frac{{a}_{u} {L}_{u} + {a}_{f} {L}_{f}}{{k}_{1} T} & ( 28 ) \\ {a}_{u} = \{ \begin{matrix}1 & {θ}_{u} = 0 \\ \frac{1 - exp ( - {k}_{2} p g {L}_{u} sin {θ}_{u} )}{{k}_{2} p g {L}_{u} sin {θ}_{u} exp ( - {k}_{2} p g {L}_{u} sin {θ}_{u} )} & {θ}_{u} \neq 0\end{matrix} & ( 29 ) \\ {a}_{f} = \{ \begin{matrix}1 & {θ}_{f} = 0 \\ \frac{1 - exp ( - {k}_{2} p g {L}_{f} sin {θ}_{f} )}{{k}_{2} p g {L}_{f} sin {θ}_{f} exp ( - {k}_{2} pg [ {L}_{u} sin {θ}_{u} + {L}_{f} sin {θ}_{f} ] )} & {θ}_{f} \neq 0\end{matrix} & ( 30 )\end{matrix}$$

In Eqn. 28, Eqn. 29, and Eqn. 30, p is blood density, g is acceleration due to gravity, and k₁ and k₂ are person-specific fitting coefficients. The hydrostatic correction factors are given by aᵤ and a_{ƒ}.

Some embodiments implement the non-autoregressive approach with purely inertial sensors for arm position tracking. When this approach was used for correcting hydrostatic pressure errors in blood pressure measurements generated with pulse transit time (using the equations above), the mean absolute error for DBP prediction was reduced from 10.6±0.6 mmHg to 6.8±0.4 mmHg (P<0.0001) in experimentation. For SBP prediction, mean absolute error was reduced from 9.6±0.6 mmHg to 5.9±0.5 (P<0.0001) in experimentation. Other approaches that include a radar-on-chip sensor for time-of-flight measurement can also be implemented.

Embodiments are used to correct hydrostatic pressure errors during ambulatory or home blood pressure monitoring. Some embodiments can be applied to both cuff-based and cuffless measurement devices. In some embodiments, a force transducer and/or temperature sensors can be implemented, and the data from these sensors can be used as input to models to further improve accuracy.

### Section 2.2

Noninvasive blood pressure (“BP”) measurement can generate several health benefits, including early detection and treatment of abnormal BP. However, conventional devices can be inaccurate when placed at different elevations relative to the heart, for example due to variations in hydrostatic pressure. Embodiments include techniques to correct hydrostatic pressure errors in noninvasive BP measurements. Embodiments track arm position, for example using wearable inertial sensors at the wrist and a deep learning model that estimates parameterized arm-pose coordinates. Arm position can then be used for analytical hydrostatic pressure compensation. An example approach uses BP measurements derived from pulse transit time, acquired using electrocardiography and finger photoplethysmography. Across hand heights of 25 cm above or below the heart, observed mean errors for diastolic and systolic BP were 0.7±5.7 mmHg and 0.7±4.9 mmHg, respectively, and did not differ significantly across arm positions. This example approach, which did not perform recalibration, can be implemented to support passive noninvasive BP monitoring, for example appropriate for everyday use.

Embodiments include techniques for cuffless noninvasive blood pressure measurement at peripheral sites on the arm, which can improve the detection of abnormal blood pressure patterns, including white-coat and masked hypertension. However, the accuracy of some emerging cuffless monitoring techniques, including those based on pulse transit time, is compromised by variations in hydrostatic pressure due to arm movement in everyday environments, limiting their clinical utility. Embodiments include techniques to track arm position using wearable inertial sensors, for example located at the wrist. A custom-derived model of pulse wave propagation demonstrates that the predicted arm-pose parameters can correct the hydrostatic pressure errors in blood pressure measurements taken across varied arm positions.

Blood pressure (BP) measurement is an informative vital sign for the diagnosis and management of many diseases (1-3), including hypertension (4, 5) and hypotension (6-8). A passive ambulatory BP measurement method that is non-intrusive and operates without user involvement could significantly improve hypertension diagnosis (9, 10). For example, white-coat hypertension and masked hypertension, which are susceptible to inaccurate measurements at the doctor's office, are by themselves estimated to constitute up to 40% of hypertension cases (11). More broadly, passive ambulatory BP measurements can enable early detection of abnormal BP patterns via repeated measurements in everyday settings (12, 13). However, noninvasive BP (“NIBP”) devices are often cuff-based, which can disruptive to the patient, for example if used over an extended period (i.e., days to weeks) (14) or during sleep (15). Further, the patient is often required to manually initiate measurement on the device, making frequent use inconvenient. These issues have motivated the development of unobtrusive and/or cuffless devices that can monitor BP in the background without prompting the patient, following initial calibration to a reference standard. The accuracy of emerging cuffless BP devices (16-20) is adversely impacted by changes in the position of the sensors relative to the heart due to differences in hydrostatic pressure (Pₕ). While also a factor in cuff-based measurement (21), error due to Pₕ is particularly impactful for cuffless approaches. In some cases, sensors are placed along the upper limb (e.g., at the wrist or finger), which can freely move relative to the heart. Without a technique to compensate for Pₕ, these cuffless BP devices will require the patient to assume the same pose for every measurement as the one used for initial calibration or require recalibration following every change in the position of the sensor-affixed limb. These limitations make accurate, long-term monitoring without prompting the patient—and hence, accurate passive BP monitoring—impractical.

To account for the Pₕ effect, embodiments track the position of the sensors relative to the heart; thus, for wearable BP sensors placed at the wrist or finger, embodiments track the upper arm and forearm positions. One established method for tracking arm position uses a fluid-filled tube that attaches to the patient at heart level and at the sensor measurement site (22); a pressure transducer inside the tube records a pressure signal, which is then used to calculate the elevation difference for Pₕ correction. Despite the simplicity, this approach is obtrusive for an everyday, wearable solution. By comparison, inertial position tracking can use tracking sensors that are small and wearable. While tracking with inertial sensors at multiple body locations has been demonstrated (23), using inertial sensors at a single location, like the wrist (as in smartwatches and fitness trackers), would be preferable. Position tracking using standard strapdown inertial navigation algorithms that involve double integration of the accelerometer signal are not suitable for arm tracking as error in the position estimate accumulate with time, typically on the order of meters one minute after initialization (24). To compensate for this error accumulation, sensor fusion techniques have been developed that include barometers to measure local air pressure, which can be then used to estimate altitude and constrain position errors (25, 26); however, these approaches are sensitive to changes in air pressure (such as moving from indoors to outdoors or using air conditioning) that can result in errors on the order of meters (27). To overcome the limitations of tracking position directly, a prior technique used an accelerometer worn at the wrist to measure forearm orientation (28). By assuming that the arm was straight, and thus that the upper arm had the same orientation as the forearm, arm position could be calculated and Pₕ artifacts corrected; however, this approach is unable to correct for Pₕ when the arm is not straight and may be confounded by antiparallel orientations for upper arm and forearm. Moreover, maintaining proper arm alignment requires patient involvement, making this method prone to user error and impractical for background monitoring. Due to these challenges, an effective general correction of Pₕ errors in BP measurements has not yet been demonstrated.

In this disclosure, a novel technique to correct Pₕ errors in BP measurements is provided that uses inertial sensors at the wrist to track upper arm and forearm positions. In some embodiments, a deep learning model estimates parameterized arm-pose coordinates to track position, and the arm-pose estimates are used as inputs to an analytical hemodynamics model to compensate for Pₕ. In some embodiments, this strategy for accurate BP measurement requires no recalibration across varied arm positions. This technique is demonstrated using BP measurements derived from pulse transit time (PTT), a cuffless technique for monitoring BP (29, 30). PTT measures the time it takes for the pressure wave to travel between two sites in the body (such as from the heart to the fingers). PTT can be acquired using electrocardiography (ECG) and photoplethysmography (PPG) by measuring the time delay between the ECG R-wave and a characteristic point in the PPG waveform. However, fluctuations in Pₕ along the path of wave propagation affect pulse wave velocity (PWV) independent of the underlying BP (31, 32). As a result, PTT measured along the upper limb varies with arm position amidst a constant central BP (33, 34), which leads to sizeable errors following arm movement. Beyond PTT-based measurement, the presented inertial arm-tracking approach may enable Pₕ correction for other BP measurement modalities. A technique to correct for Pₕ without recalibration would allow cuffless NIBP monitoring technologies to take repeated BP measurements accurately across different body positions without intervention from the patient, corresponding to conditions of everyday use

### Results

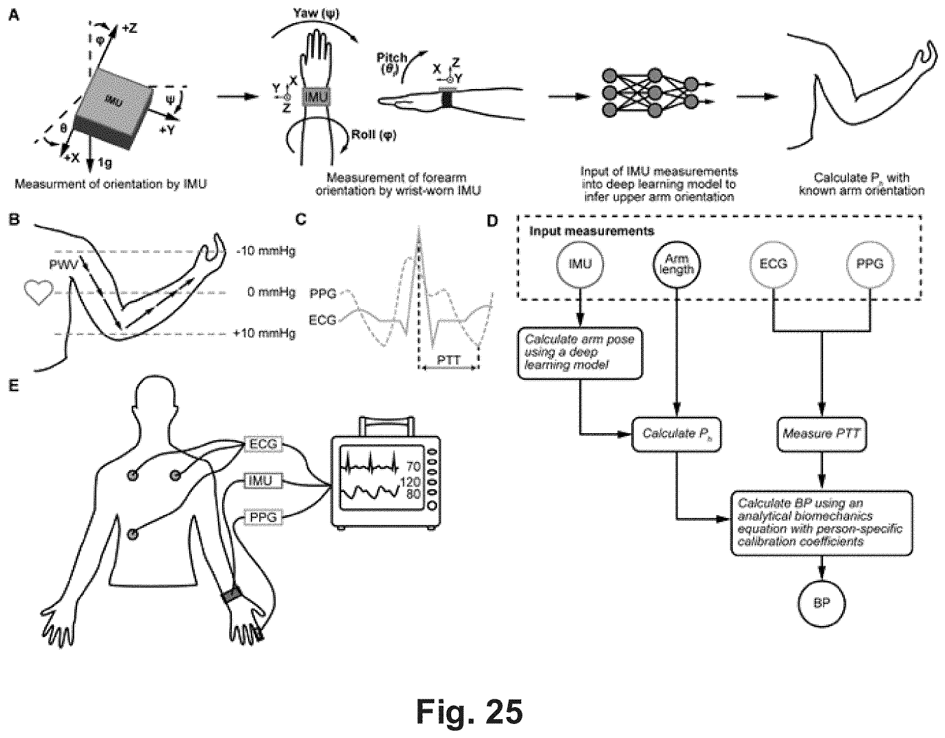

BP measurement from PTT: Overview of approach. Embodiments include a deep learning-assisted method to correct errors due to Pₕ in noninvasive BP measurements. First, a deep learning model can estimate arm pose using a sequence of measurements from, for example, a wrist-based inertial measurement unit (IMU), which can include an accelerometer, and optionally a gyroscope and/or magnetometer. This estimated pose and measured arm length can then be used to calculate Pₕ for subsequent correction, as illustrated in FIG. 25, Panel A. An evaluation of such an approach can be performed using BP derived from PTT, which is affected by variations in PWV that arise from changes in Pₕ, as illustrated in FIG. 25, Panel B. In an example implementation, PTT is calculated as the delay between the ECG R-wave peak and the onset time of the finger PPG waveform, defined as when the second derivative is maximized (as illustrated in FIG. 25, Panel C). While any fiducial point can be selected as the distal time reference, PTT calculated using PPG onset has been shown to correlate better to BP (35). Finally, in some embodiments the calculated Pₕ and measured PTT serve as inputs into an analytical model of pulse wave propagation to predict BP. The block diagram detailing an example pipeline is shown in FIG. 25, Panel D. The approach is demonstrated using data collected from 20 human participants, with intake data summarized in Table S1. The devices in this sample implementation include one lead ECG, finger PPG, and wrist-mounted IMU (as illustrated in FIG. 25, Panel E)

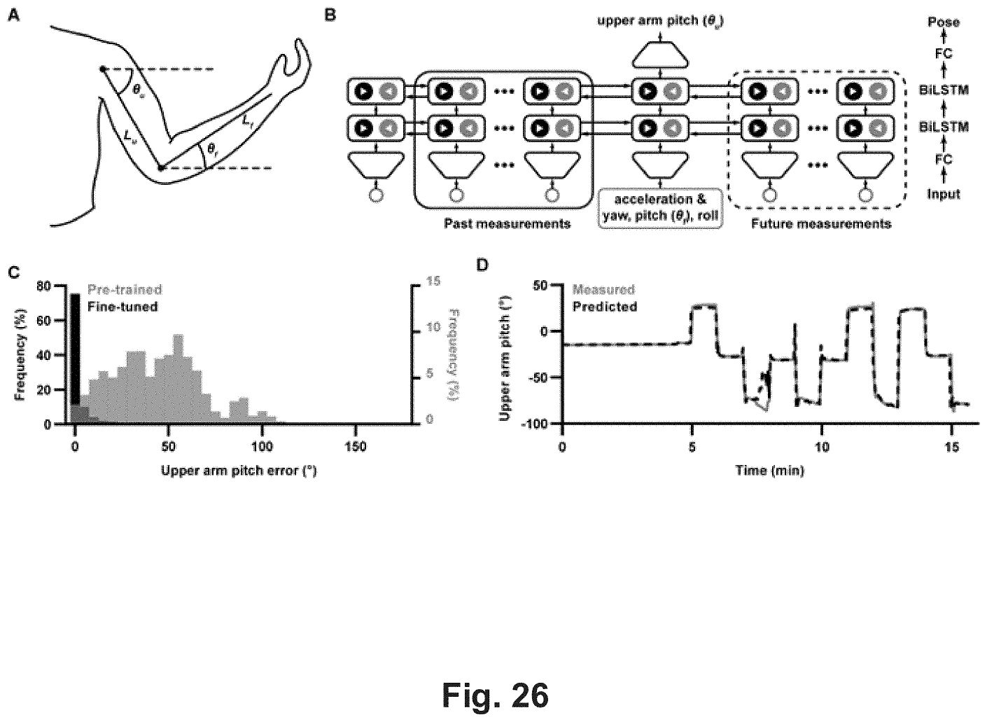

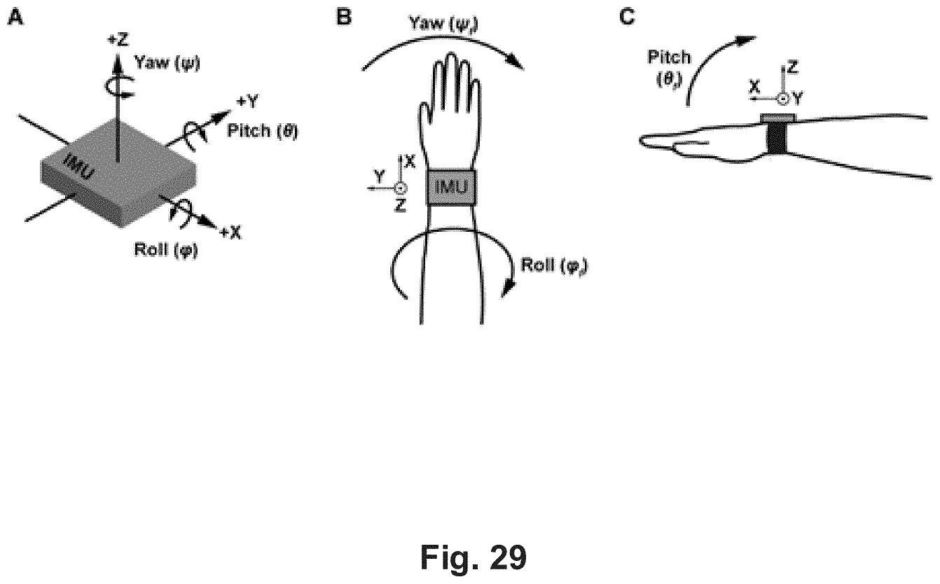

Deep learning enables arm posing. In one approach for tracking relative elevation along the arm, the arm can be considered as two rigid segments, the upper arm and forearm, with lengths Lᵤ and L_{ƒ}, respectively (as illustrated in FIG. 26, Panel A). The shoulder can be considered the zero-Pₕ reference point instead of the heart to simplify calculations such that relative elevation is parameterized by the angles between the arm segments and the horizontal axis, θᵤ and θ_{ƒ} This simplification is reasonable as the height difference between the heart and shoulder is small such that the Pₕ difference is minimal. If arm length is known, tracking relative elevation thus reduces to tracking these two angles, here referred to as the arm pose. To minimize additional instrumentation, in some embodiments pose is tracked using a single IMU, capable of directly measuring linear acceleration and orientation, for example at the wrist. The IMU can be attached to the wrist, for example with the positive x-axis pointing down the arm such that, after converting the measured forearm orientation to intrinsic ZYX Tait-Bryan angles (yaw, pitch, and roll), the pitch can be equal to θ_{ƒ} (as illustrated in FIG. 29). For upper arm tracking, a deep learning model (with some similarities to Deep Inertial Poser (36)) can be implemented that uses a two-layer, bidirectional (37) Long Short-Term Memory (38) (“LSTM”) model to predict upper arm orientation given the current frame of forearm orientation and acceleration along with 19 past and 5 future frames (as illustrated in FIG. 26, Panel B). Orientation can be represented as a unit quaternion that describes rotation from the sensor-local frame to the global inertial frame. Acceleration can be given in the global inertial frame with gravity canceled. The predicted upper arm orientation can then be used to calculate θᵤ.

In some embodiments, a deep learning model can be trained (e.g., pre-trained) e.g., using the Virginia Tech Natural Motion Dataset (39) (VT-NMP) and fine-tuned on data collected using implemented IMUs. Fine-tuning can be used to account for the subtle differences in the distribution between the datasets and to condition the model on the types of motion encountered during inference (36). In other embodiments, a deep learning model can be trained based on data collected using implemented IMUs, or any other suitable collected data and/or data sets. Model evaluation can be performed using a leave-one-out cross-validation method (40, 41) where data from 19 participants can be used for fine-tuning while data from the remaining participant can be used for testing, and the procedure repeated until each participant is tested. In an example implementation, the error histograms calculated on the test fold pooled across the 20 study participants (as illustrated in FIG. 26, Panel C) demonstrate that fine-tuning is useful, reducing the angular error between the measured reference and predicted θᵤ from 44.2±24.6° to 4.5±11.2°. Though more/different types of collected data may perform well without pre-training and/or fine-tuning. The time series of 0, for a representative participant (as illustrated in FIG. 26, Panel D) shows that the prediction closely tracked the reference. While there are occasional deviations, they occur predominately during sudden movement, and the predicted signal reconverges to the ground truth. These results demonstrate that arm pose can be tracked using a single IMU sensor at the wrist (for our BP correction purposes).

Modeling the effect of arm pose on PTT. Starting from the Moens-Korteweg (42) and Hughes (43) equations, an ordinary differential equation can be derived to model pulse wave propagation as a function of arm pose and BP. By considering arm pose to be constant for a cardiac cycle, the equation can be integrated, yielding a model for PTT (7) as a function of BP (P) and arm pose:

$$\begin{matrix}T = {α}_{u} {T}_{u} + {α}_{f} {T}_{f} = {α}_{u} ( \frac{{L}_{u}}{{k}_{1} exp ( {k}_{2} P )} ) + {α}_{f} ( \frac{{L}_{f}}{{k}_{1} exp ( {k}_{2} P )} ) & [ 31 a ] \\ {α}_{u} = \{ \begin{matrix}1 , & {θ}_{u} = 0 \\ \frac{1 - exp ( - {k}_{2} ρ {gL}_{u} sin {θ}_{u} )}{{k}_{2} ρ {gL}_{u} sin {θ}_{u} exp ( - {k}_{2} ρ {gL}_{u} sin {θ}_{u} )} , & {θ}_{u} \neq 0\end{matrix} & [ 31 b ] \\ {α}_{f} = \{ \begin{matrix}1 , & {θ}_{f} = 0 \\ \frac{1 - exp ( - {k}_{2} ρ {gL}_{f} sin {θ}_{f} )}{{k}_{2} ρ {gL}_{f} sin {θ}_{f} exp ( - {k}_{2} ρ g [ {L}_{u} sin {θ}_{u} + {L}_{f} sin {θ}_{f} ] )} , & {θ}_{f} \neq 0\end{matrix} & [ 31 c ]\end{matrix}$$

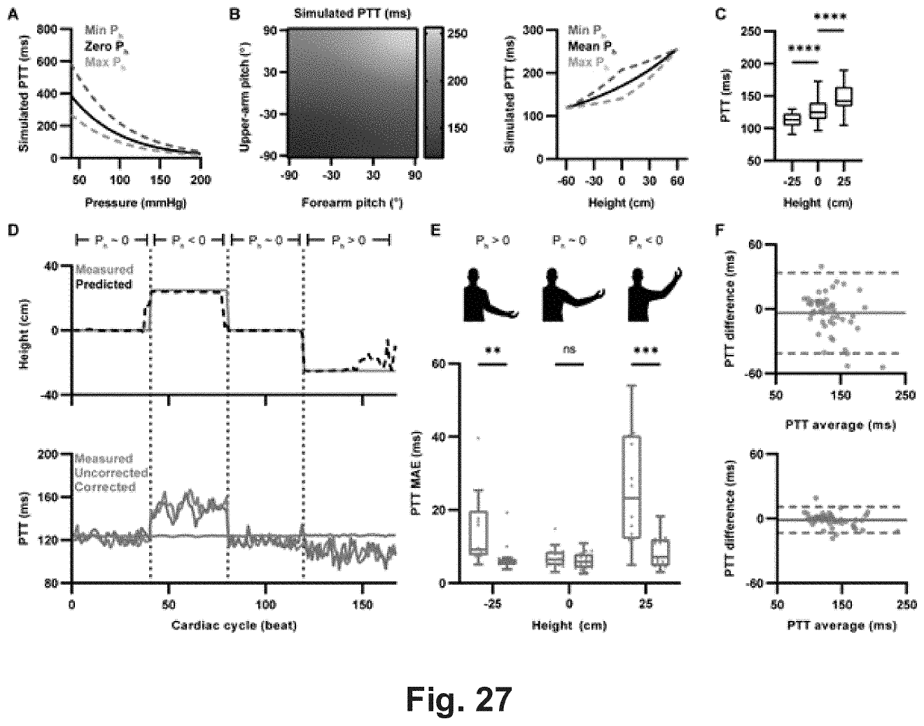

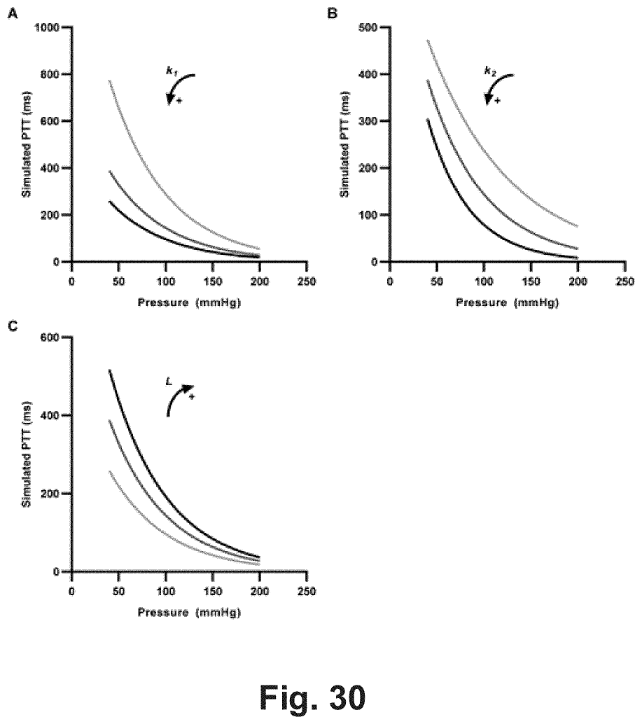

In the above, ρ is blood density, g is acceleration due to gravity, and k₁ and k₂ are person-specific fitting coefficients (see the “Mathematical model” section in Methods for the full derivation). Inspection of these equations shows that PTT is a sum of two uncorrected partial transit times, describing travel along the upper (Tᵤ) and lower arm (T_{ƒ}), weighted by correction factors, αᵤ and α_{ƒ}, that compensate for the effects of Pₕ. Example simulations (as illustrated in FIG. 27, Panel A and FIG. 30) revealed that, for a fixed arm pose, PTT decreases monotonically with increasing BP; however, changes in arm pose were found to cause substantial variation in predicted PTT for a given pressure, with the effect minimized for large pressures.

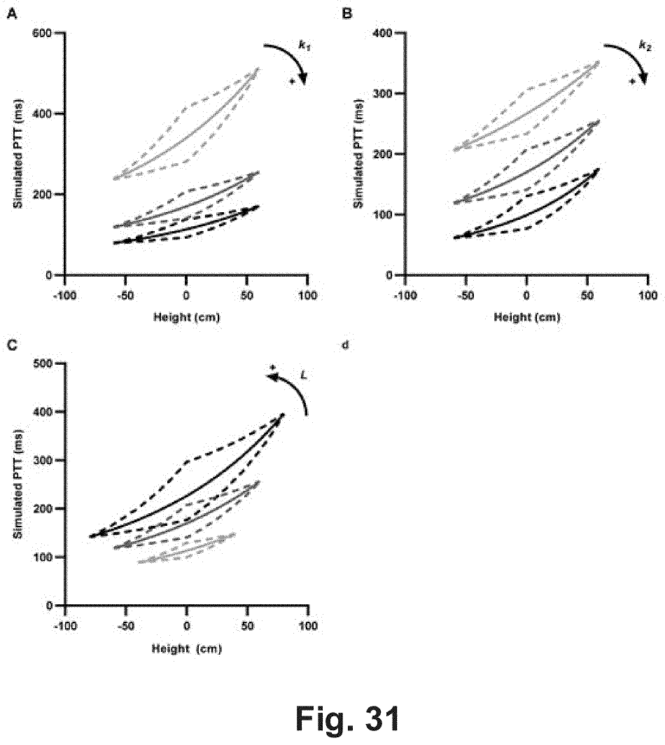

To further investigate the impact of arm pose, PTT can be simulated over a grid of arm pose specifications with constant pressure (as illustrated in FIG. 27, Panel B). In the simulation an embodiment of the model predicted an increase in PTT with increasing arm pitch (indicating moving the limb upward), as this movement causes a decrease in Pₕ and a subsequent decrease in PWV. Conversely, the simulations showed a decrease in arm pitch resulted in a decrease in PTT. As controlling both θᵤ and θ_{ƒ} in an experimental setting is challenging, we also explored the effects of Pₕ on PTT due to the relative height of the distal measurement site (h) alone (as illustrated in FIG. 27, Panel B and FIG. 31). Based on these simulations, PTT tends to increase with increasing h; however, an embodiment of the model predicts an asymmetric, nonlinear relationship where an increase in h causes a larger increase in PTT than the equivalent decrease in h. The simulations also demonstrate the usefulness of using arm pose, rather than h alone, for Pₕ compensation. In particular, as arm pose has two degrees of freedom (θᵤ and θ_{ƒ}), PTT can vary independently of central pressure even when h is fixed. As a result, the simulations show a distribution of PTTs for a given h and fixed BP that is widest when h=0 cm and narrow at the extremes.

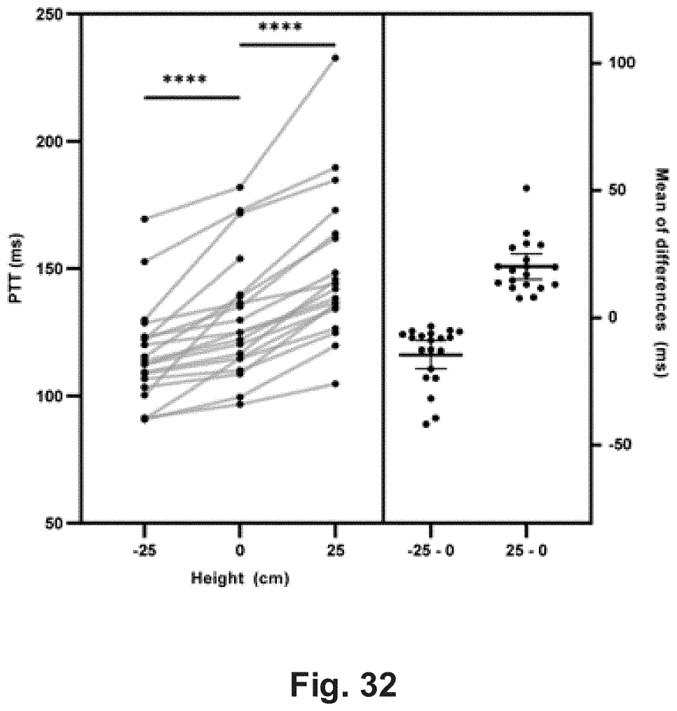

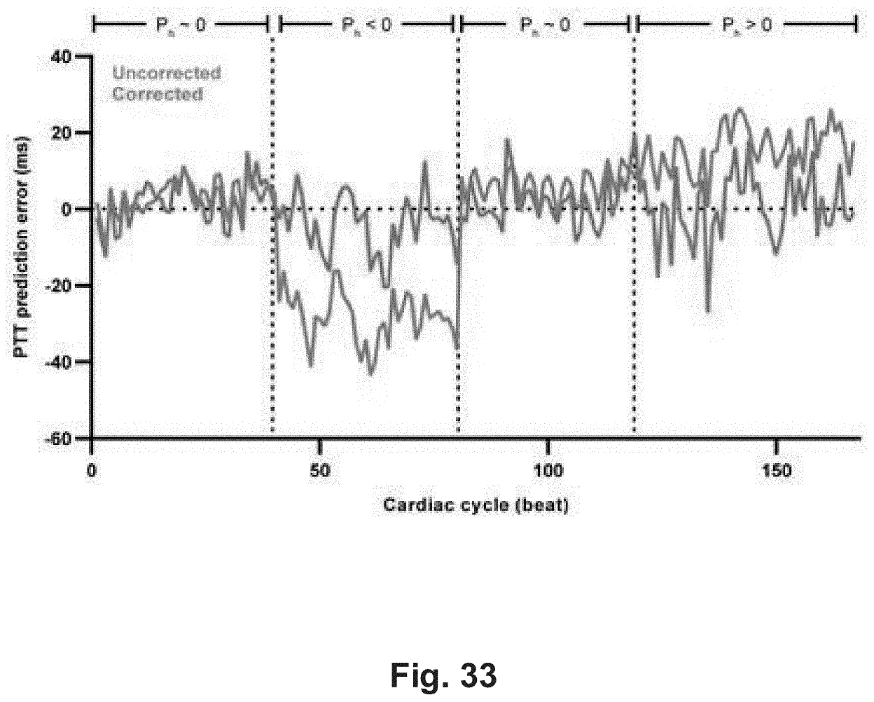

Experimental evaluation of analytical model for wave propagation. To validate the embodiment of the mathematical model, PTT was recorded in 20 participants as they moved their arm through a sequence of movements to vary h between −25, 0, and 25 cm. Matching the simulations, a decrease in h resulted in a significant decrease in PTT (P<0.0001) while an increase in h resulted in a significant increase in PTT (P<0.0001) (as illustrated in FIG. 27, Panel C and FIG. 32). Next, it was evaluated how well the embodiment of the model “corrected” for Pₕ effects fit the measured PTTs, compared to an “uncorrected” model that fixed αᵤ and α_{ƒ} to 1 and thus did not account for hydrostatic effects. For each participant, the measured data were divided into two non-overlapping windows, one for personalized calibration and the other for testing. The calibration data were then used to find the person-specific fitting coefficients for the corrected and uncorrected models by minimizing the mean squared error between the measured and predicted PTT with mean arterial pressure as P. For the calibration data, the height-tracking time series for a representative participant (as illustrated in FIG. 27, Panel D) demonstrated that predicted pose from the deep learning model could track hand elevation ground truth. The beat-to-beat, best-fit PTT predictions (as illustrated in FIG. 27, Panel D and FIG. 33) indicated that the uncorrected baseline failed to track the measured PTT, with an increased error when h was non-zero. By comparison, the corrected model closely tracked the measured PTT for the entire calibration interval (as illustrated in FIG. 27, Panel D and FIG. 33).

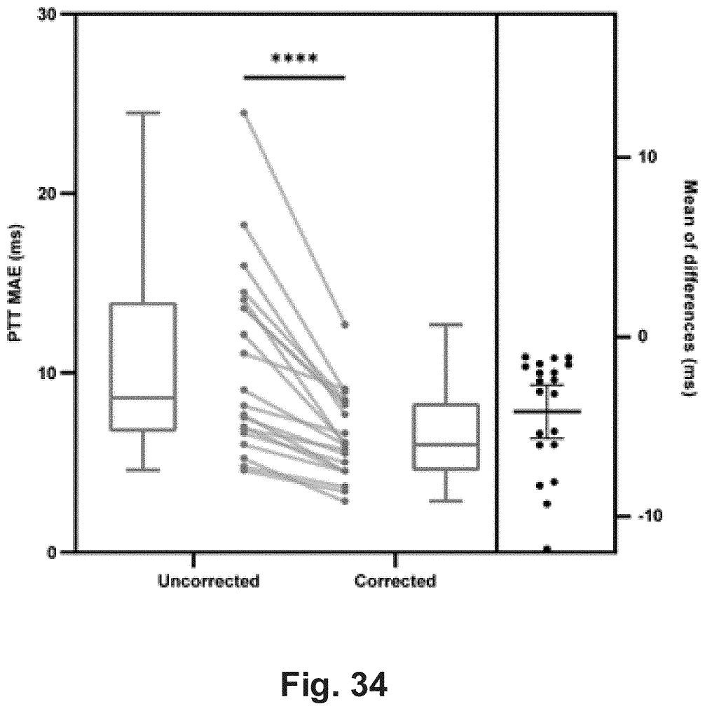

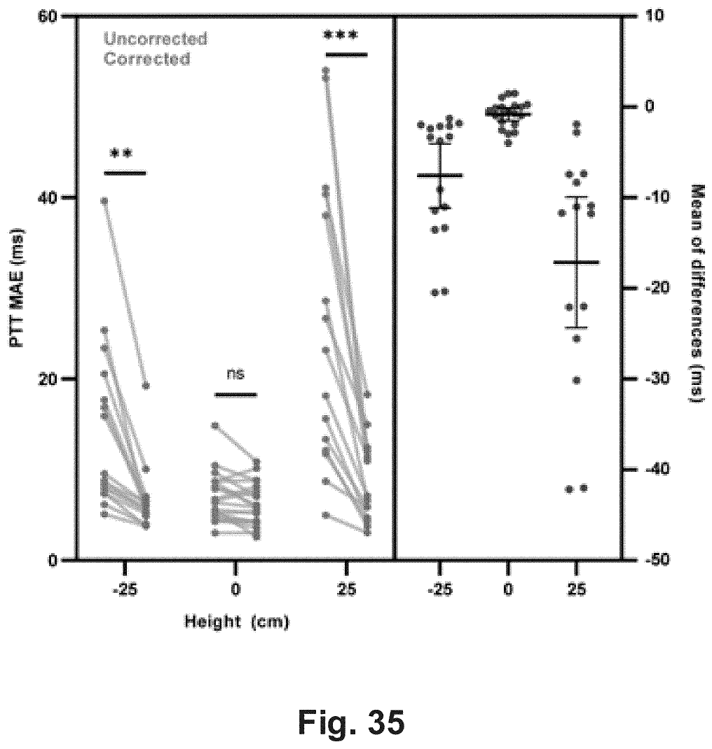

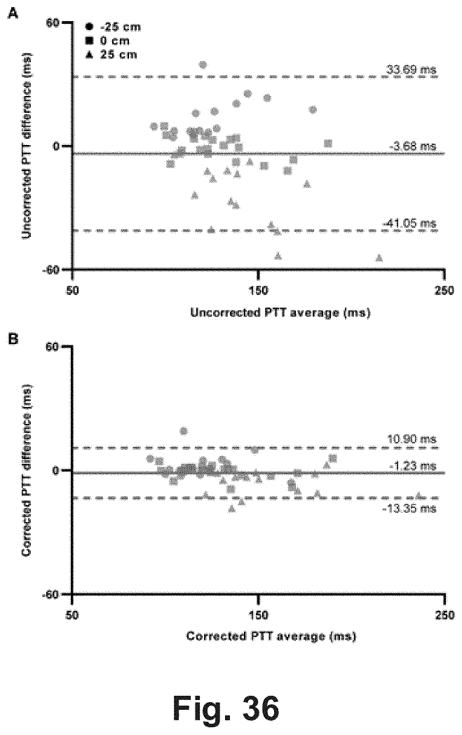

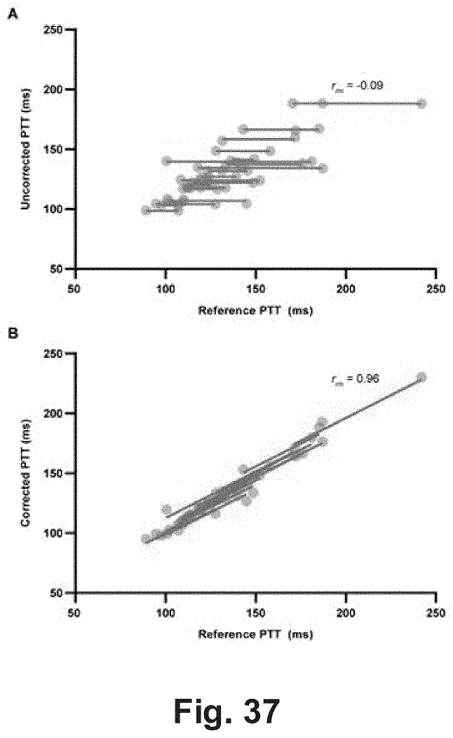

Next, embodiments of the model fit for the calibration data across all participants was assessed, with results summarized in Table 1. The pose-correction significantly reduced the mean absolute error (MAE) between measured and best-fit PTT (as illustrated in FIG. 34), with an average improvement of 4.16±0.71 ms (P<0.0001). Reduced MAE was observed for each h (as illustrated in FIG. 27, Panel E and FIG. 35), with an average improvement of 7.6±1.7, 0.8±0.3, and 17.2±3.4 ms for h=−25, 0, and 25 cm, respectively (P=0.0012, P=0.0708, and P=0.0005). Significant reduction in MAE for h=25 cm and h=−25 cm was expected, as the uncorrected baseline is unable to account for the Pₕ-induced change in PTT that follows a change in hand height. Bland-Altman plots comparing the measured and best-fit estimates for PTT (as illustrated in FIG. 27, Panel F and FIG. 36) indicate that pose correction improves both bias and precision, resulting in a tightening of the 95% limits of agreement (“LoA”). In addition to improving absolute fit and agreement, the pose correction significantly improved the correlation of the best-fit PTT predictions to the measured PTT, with the repeated measures correlation increasing from −0.09 to 0.96 (P<0.0001) (as illustrated in FIG. 37). As shown by improved absolute fit and agreement, the corrected-pose equations fit the measured data.

Blood pressure predictions using pose-corrected model. The embodiment of the model was next evaluated when used for BP prediction. By rearranging Eqn. 31 to solve for BP, the following pose-corrected BP model can be shown:

$$P = \frac{1}{{k}_{2}} ln \frac{{α}_{u} {L}_{u} + {α}_{f} {L}_{f}}{{k}_{1} T} \qquad [ 32 ]$$

Two separate BP models for diastolic (DBP) and systolic pressure (SBP) were applied. For personalized calibration, the mean squared error was minimized between the calibration subset of the measured BP and the predictions from the pose-corrected BP model to obtain person-specific values for the coefficients k₁ and k₂. The model embodiment was then evaluated on the held-out test data not seen during calibration. To assess the impact of pose correction, the accuracy of BP estimates generated with the Pₕ-compensated “corrected” model were compared with that of an “uncorrected” model (i.e., with αᵤ and α_{ƒ} set to 1).

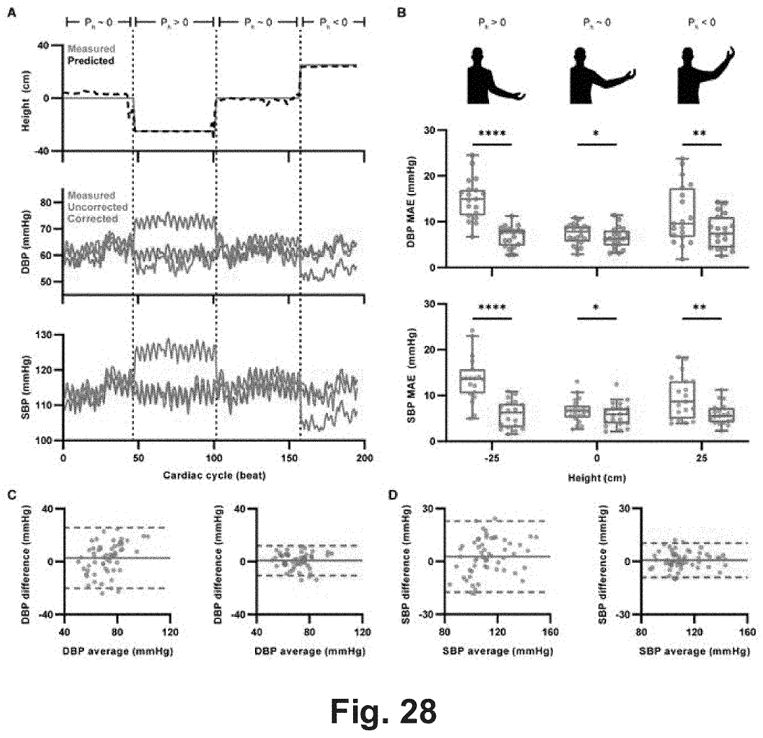

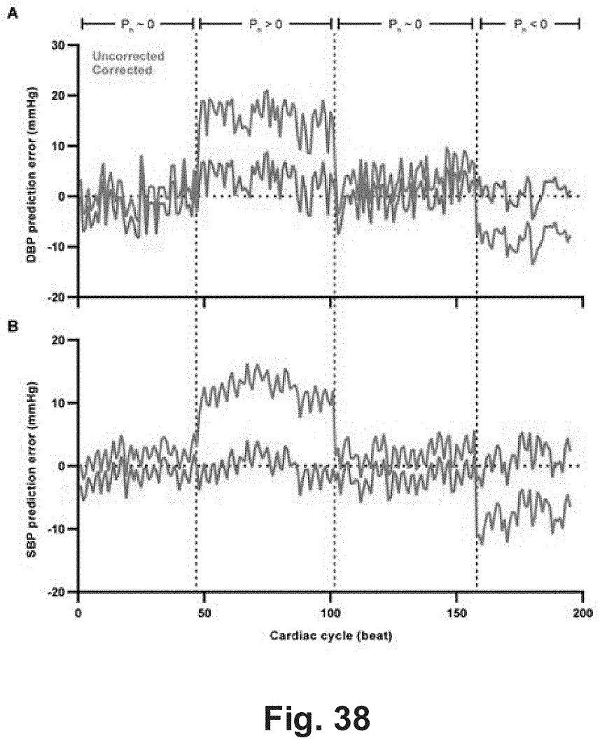

For a representative participant, the relative height time series (as illustrated in FIG. 28, Panel A) for the test set indicated that the embodiment of the deep learning model tracked h. From the beat-to-beat pressure predictions (as illustrated in FIG. 28, Panel A and FIG. 38), it was found that the predictions generated from the uncorrected and corrected models were comparable when h=0 cm, as θᵤ and θ_{ƒ} were both near 0° and thus minimized hydrostatic effects. However, predictions for both DBP and SBP made using the uncorrected model tended to underestimate the measured reference when h=25 cm and overestimate when h=−25 cm. With pose-correction, the pressure predictions more closely tracked the reference for both conditions.

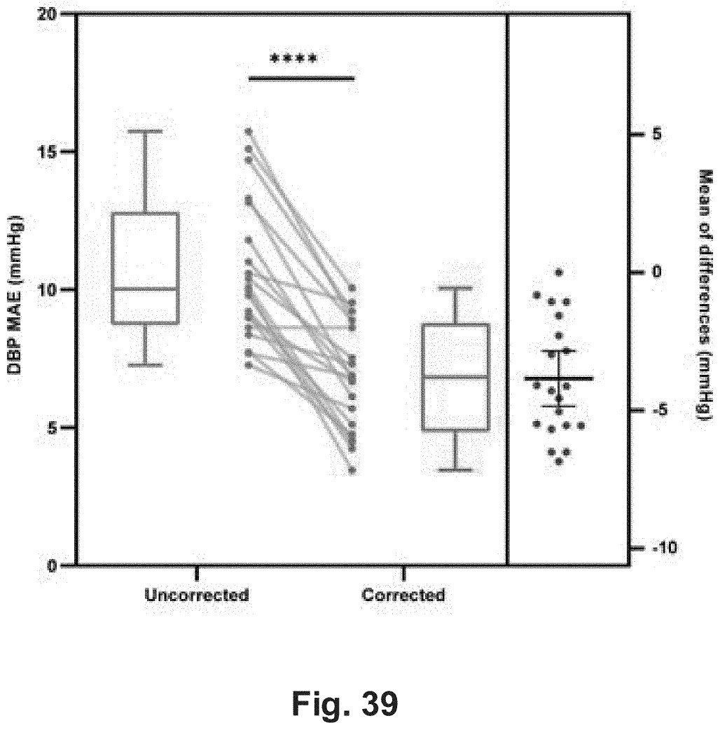

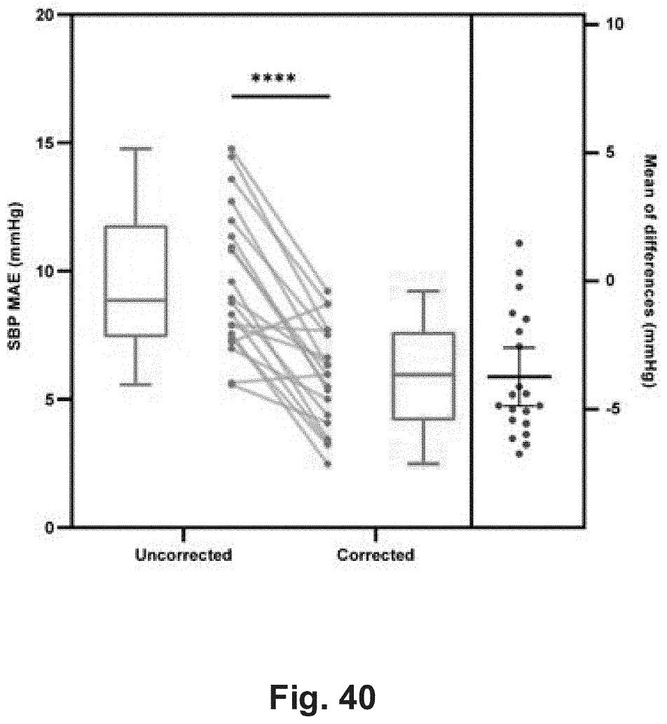

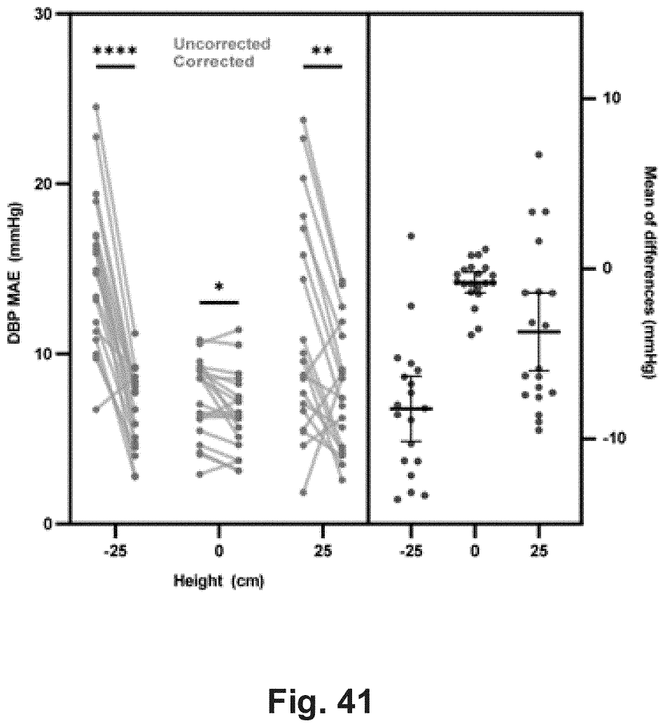

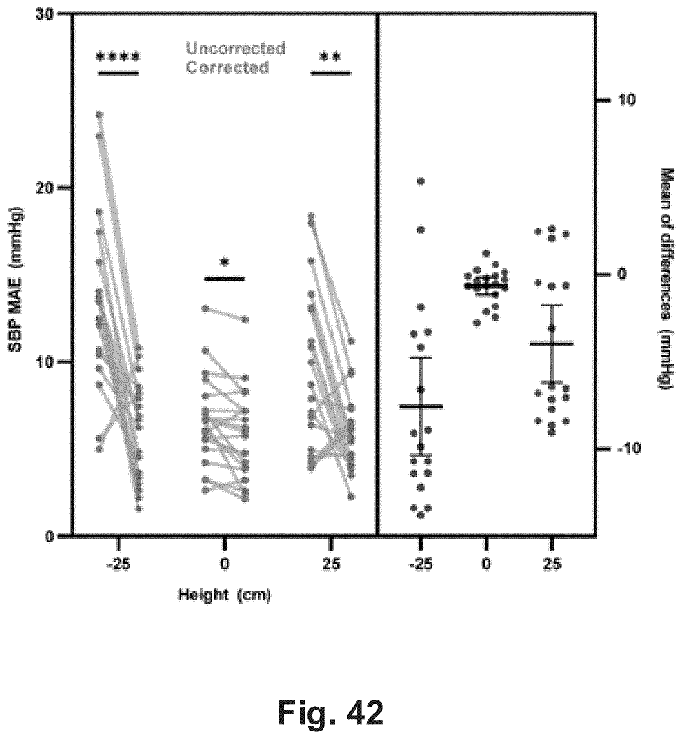

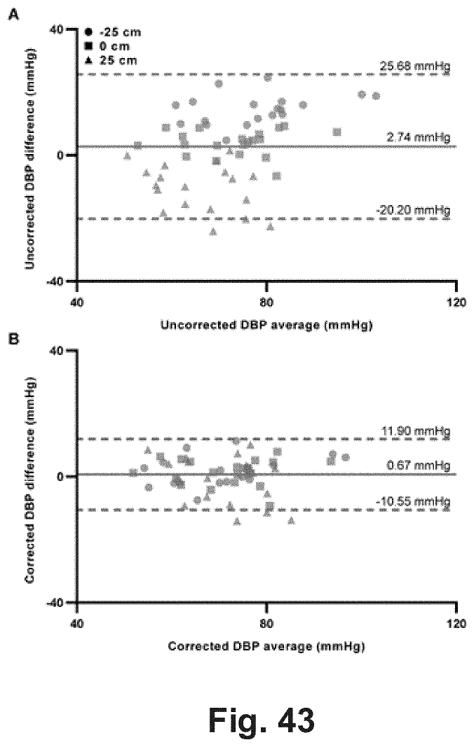

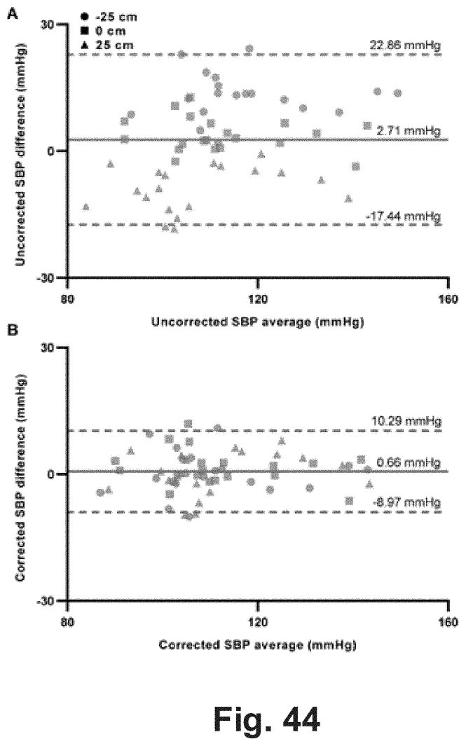

Across all participants, the embodiment of the corrected model significantly reduced overall MAE to 6.8±0.4 (P<0.0001) and 5.9±0.5 mmHg (P<0.0001) for DBP and SBP, respectively (as illustrated in FIGS. 39 and 40). Improvement in MAE was consistent across h for DBP predictions (as illustrated in FIG. 28, Panel B and FIG. 41), with MAE reduced to 6.8±0.5, 6.5±0.5, and 7.8±0.8 mmHg for h=−25, 0, and 25 cm, respectively (P<0.0001, P=0.0356, and P=0.0096). Reduced MAE was also found at each h for SBP prediction (as illustrated in FIG. 28, Panel B and FIG. 42), with MAE reduced to 5.9±0.7, 5.8±0.6, and 5.9±0.5 mmHg for h=−25, 0, and 25 cm, respectively (P<0.0001, P=0.0284, and P=0.0051). No significant difference in MAE across the different values of h was observed when using the corrected model for DBP (P=0.2217) or SBP (P=0.9906) prediction. These results are summarized in Table 2. In addition to reduced MAE, the embodiment of the corrected model improved agreement. The Bland-Altman plots for the measured reference pressure compared to the model predictions (as illustrated in FIG. 28, Panels C and D, and FIGS. 43 and 44) revealed that inclusion of the pose correction improved both the bias and precision of the pressure estimates, resulting in greater agreement as indicated by the tightening of the 95% LoA. The mean error for DBP and SBP prediction was reduced to 0.7±5.7 mmHg and 0.7±4.9 mmHg, respectively, which meets the accuracy criteria for the Association for the Advancement of Medical Instrumentation/European Society of Hypertension/International Standards Organization (AAMI/ESH/ISO) validation standard (44). Taken together, the embodiment of the corrected model is able to compensate for Pₕ effects without recalibration, enabling accurate BP prediction under conditions of varying h induced by changes in arm position.

Discussion

Embodiments that implement a cuffless technique for arm BP measurement are the first known approach that corrects Pₕ errors by independently considering the position of the upper arm and forearm. Arm position can be tracked in a parameterized arm-pose coordinate system where a wrist-worn IMU measures θ_{ƒ} and a deep learning model uses forearm acceleration and orientation from the IMU to infer θᵤ. Further, an analytical model for PTT as a function of BP and arm pose; applied in an inverse manner is derived, an embodiment of the model reported corrected estimates of BP, given measures of PTT and arm pose, with bias and precision meeting clinical accuracy criteria and with no significant difference in accuracy across arm position that varied hand elevations. By correcting Pₕ errors induced by varying the relative elevation of the finger PPG sensor and improving BP prediction accuracy, embodiments can achieve accurate cuffless NIBP monitoring from PTT (without the need for recalibration in some implementations).

PTT-based approaches are well-suited for cuffless arm BP tracking for patients in the hospital, as this technology can leverage the ECG and finger PPG signals already routinely monitored in critically ill patients (45-48) for unobtrusive PTT calculation while a wrist-mounted IMU compensates for Pₕ. By comparison, current devices for BP measurement in critically ill patients, including cuff-based devices that obtain frequent measurements and could disturb the user during the nighttime period (15), do not correct for Pₕ fluctuations caused by changes in the location of the patient's arm relative to the heart. Embodiments can also be adapted for monitoring BP in patients outside of the hospital, including in an office setting or at home, by using unobtrusive wireless ECG electrodes and PPG sensors that build on advances in wireless physiological monitoring (49) to calculate PTT. Further, many commercially available smartwatches already contain ECG electrodes and PPG sensors in addition to inertial sensors (e.g., accelerometer, gyroscope, and magnetometer) used in embodiments. Hence, the techniques presented herein could be applied to current ECG-capable smartwatches for intermittent measurement, and other devices could leverage single-arm ECG (50, 51) to enable passive monitoring without patient involvement. In contrast to current devices and competing PTT techniques for arm BP measurement where accuracy relies on the patient maintaining a fixed arm position, the Pₕ correction implemented by embodiments enables accurate BP estimation across varied arm positions (e.g., without recalibration). This capability addresses an problem experienced with other cuffless devices, particularly for BP monitoring at home, since patients may not adhere to keeping their arms in a fixed position during BP measurement. Thus, the presented approach to cuffless BP monitoring is better suited for application in free-living, everyday environments outside a clinical setting.

### Materials and Techniques

Design. Embodiments compensate for the effects of Pₕ using wearable inertial sensors (e.g., at the wrist) to enable accurate BP prediction across different arm positions without requiring recalibration. To validate the techniques, a human study was performed under protocols approved by the Institutional Review Boards at Columbia University Medical Center (IRB no. AAAR5932) and with written, informed consent from all participants. Adult participants were recruited from the students and staff population at Columbia University Medical Center. The exclusion criteria were a history of (1) Raynaud's phenomenon or vascular disease involving the upper extremities, (2) history of cardiovascular disease, (3) uncontrolled hypertension, and (4) pregnancy. Sex, age, BMI, and other parameters in our sample population reflected the natural distribution among students and staff at Columbia University. Summary of intake data for the recruited participants is found in Table S1.

The study fit participants with noninvasive sensors (specified in the Data Collection section) and guided them through various arm movements, with the resulting data recorded for analysis. A deep learning model was trained for arm position tracking. The deep learning model was pre-trained using the VT-NMP dataset (39). Fine-tuning and testing of the model was then performed using the inertial sensor data from the human participants. Other embodiments can leverage a model trained with observed or other data sets. Next, an analytical equation of PTT under the effects of varying Pₕ was derived and computationally modeled to understand how arm position affects PTT. A subset of the data collected from each participant was used to experimentally validate the derived PTT model. Finally, we evaluated the performance of this analytically derived model for BP prediction across varied arm positions. A subset of data was used for personalized calibration of the BP prediction model for each participant. The calibrated models were then evaluated on the remaining held-out data for each participant. Investigators were not blinded during the study. Sample size (n=20) was chosen according to a cuffless BP validation standard (IEEE 1708a-2019) that specifies 20 participants for the initial pilot phase (52).

Data Collection. In one implementation, participants were fit with various noninvasive sensors and devices including: continuous noninvasive BP measurement device (BIOPAC NIBP), ECG electrodes (3M Red Dot 2237), two PPG sensors (BIOPAC TSD200), and two IMU devices that contained a combination accelerometer/gyroscope (STMicroelectronics LSM6DSM), magnetometer (STMicroelectronics LIS2MDL), and motion coprocessor (EM Microelectronics EM7180). Other embodiments can implement any other suitable sensors and devices. Additionally, arm length (from humeral head to radial styloid with arm abducted to 90° with elbow extended with thumb facing up) and hand length were recorded for each participant. The BIOPAC NIBP was operated in a contralateral setup where the upper arm cuff was attached to the participant's non-dominant arm and the two finger cuffs were placed around the index and middle fingers of the dominant hand. Prior to initial calibration, both arms were placed on armrests located at heart level. The upper arm cuff was then used to acquire an initial BP measurement while the finger cuffs acquired subsequent beat-to-beat BP measurements. The dominant arm with the finger cuffs was maintained at heart level for the duration of the experiment. The ECG electrodes were placed in Lead II configuration, and the signal was fed into an amplifier (BIOPAC ECG100C). The PPG sensors were attached to the ring finger of each hand and fed into separate amplifiers (BIOPAC PPG100C). The output from these sensors was recorded using a digital acquisition system (BIOPAC MP160) at 2,000 Hz. The two IMUS were affixed to the upper arm and the wrist of the non-dominant arm. The sensors were aligned such that the sensor-frame positive x-axis pointed down the arm. The sensors were controlled using a microcontroller (PJRC Teensy 3.6). Data was collected at 100 Hz and logged to a microSD card. Synchronization of data acquisition was accomplished using a digital output signal from the MP160.

After an initial 5-minute period of rest to acquire baseline readings (with both arms at heart level), the participants were asked to raise or lower their non-dominant arm to the designated armrest, located 25 cm above or below heart level, to cause a Pₕ perturbation. The participant was instructed to rest at the new position for 1 minute. After the minute had elapsed, the participants were instructed to move their non-dominant arm to another position with a 1-minute rest. This procedure was repeated until the participant had completed a sequence of 11 movements to one of three armrests (25 cm below heart level, heart level, or 25 cm above heart level). Participants were randomly assigned to one of two movement sequences, either [0, 25, 0, −25, 0, −25, 0, 25, −25, 25, 0, −25] or [0, −25, 0, 25, 0, 25, 0, −25, 25, −25, 0, 25], with the first height indicating the initial rest period. After the sequence of arm movements was completed, the data was saved for downstream analysis.

IMU data preprocessing. For in-house IMU data, acceleration and orientation were rotated from the North-East-Down inertial frame to the North-West-Up frame. The data was then filtered using a 3^{rd} order Savitzky-Golay filter with a window size of 51 samples, and the resulting data was downsampled to 40 Hz. For the VT-NMP dataset, a yaw rotation of 90° was applied to acceleration and orientation to align the measurements with our coordinate system (i.e., positive x-axis points down the arm), and the resulting data was downsampled to 40 Hz. Acceleration was represented in the global inertial frame with gravity cancelled. Orientation was represented as a unit quaternion. Pitch (i.e., rotation around the intrinsic y-axis) was extracted after converting to intrinsic ZYX Tait-Bryan angles.

Deep learning model architecture and training. The embodiment of the deep learning model was implemented in Python with the PyTorch (53) library and was trained on an Nvidia RTX 2080 Super GPU. Pose prediction was performed using a modified implementation of the Deep Inertial Poser (36) architecture. The implemented model had four layers: (1) a time-distributed fully connected layer with 256 units, (2) a bidirectional LSTM layer with 256 units, (3) a second bidirectional LSTM layer with 256 units, and (4) a time-distributed fully connected layer with 4 units used for upper arm orientation prediction. The predicted quaternion was then normalized to unit norm. Finally, the quaternion was used to calculate Bu. The input to the model was a sequence of forearm orientation and acceleration. The output was the corresponding sequence of θᵤ. During pre-training and fine-tuning, the model was used in the many-to-many scheme, with the loss minimized over the full prediction sequence. During inference, a single θᵤ was predicted for each input sequence, as shown in FIG. 26, Panel B.

For pre-training with the VT-NMP dataset, the same training and validation split proposed by Geissinger and Asbeck (54) was used. For each participant, the full motion sequence was used to construct batches of partially overlapping sub-sequences using a sliding window of length 25 with a stride of 15. A yaw rotation is then applied to each window such that the orientation for the first sample has a yaw of 0°. The model weights were randomly initialized and trained to minimize the MAE loss by stochastic gradient descent using the AdamW (55) optimizer for 5 epochs with batch size of 512, an initial learning rate of 1e-03, and a weight decay of 0.03. During training, the learning rate was decayed using the cosine annealing scheduler. To prevent overfitting, validation loss was monitored to implement early stopping. Model hyperparameters were optimized based on performance on the validation split.

For fine-tuning and model evaluation, a leave-one-out cross-validation technique (40, 41) was used. The IMU data was split into 20 folds, with each fold comprising the data from a single participant. For each iteration of cross-validation, data from 19 folds were used for fine-tuning while data from the remaining fold was used for testing. During fine-tuning, the first 90% of data from each participant was used for training while the remaining 10% was used for validation. Data was pre-processed into partially overlapping sub-sequences, as described previously. The model weights were initialized using the weights from the model pre-trained on VT-NMP dataset. The model was trained by stochastic gradient descent using the AdamW optimizer for 5 epochs with batch size of 512, an initial learning rate of 1e-04 and a weight decay of 0.03. Learning rate was decayed using the cosine annealing scheduler. To prevent overfitting, validation loss was monitored to implement early stopping. During testing, sub-sequences were created from the held-out fold using a sliding window of length 25 with stride 1. The input sequences were passed through the implemented model to generate sequences of predicted θᵤ. A single pose corresponding to the current timestep (i.e., the 20^{th} frame) was extracted from the prediction sequence. Pitch time series were then reconstructed by concatenating all predictions for that participant. This procedure was repeated until every fold was used for testing.

Mathematical Model. Arm PTT is a measure of the time it takes for the BP pulse wave to travel from the heart to a distal measurement site. It is thus a function of the velocity of this wave (PWV) and arm length (L). By the Moens-Korteweg equation (42), PWV (c) can be related to the elastic modulus (E), vessel thickness (h), vessel diameter (d), and blood density (ρ) as follows:

$$c = \sqrt{\frac{E h}{ρ d}} . \qquad [ 33 ]$$

By the empirically derived Hughes equation (43), the elastic modulus is exponentially related to the transmural pressure (Pₜ):

E=E₀e^{αP}^{ₜ}. [34]

Here, E₀ is the elasticity when Pₜ is zero and α is a constant. Substituting the Hughes equation into the Moens-Korteweg yields an expression for c as a function of Pₜ:

$$c = \sqrt{\frac{{E}_{0} h {e}^{α {P}_{t}}}{ρ d}} . \qquad [ 35 ]$$

If the ratio \sqrt{E₀h/ρd} is assumed constant, this equation can be simplified by defining new constants, k₁=\sqrt{E₀h/ρd} and k₂=½ α, as follows:

c=k₁·e^{k}^{₂}^{·P}^{ₜ}. [36]

Thus, as the wave travels to the distal site, PWV varies dependent on the transmural pressure at each point along the arm. To account for these variations, first substitute in the definition for Pₜ:

c=k₁·e^{k}^{₂}^{(P}^{ₐ}^{-P}^{ₑₓₜ}⁾, [37],

In the above, Pₐ is the intra-arterial pressure and Pₑₓₜ is external pressure. Next, we assume pulse pressure amplification is negligible along the arm such that Pₐ=P. Further, we assume Pₑₓₜ is dominated by Pₕ. Substituting for Pₐ and Pₑₓₜ yields a model for PWV as a function of BP and Pₕ:

c=k₁·e^{k}^{₂}^{(P+P}^{ₕ}⁾. [38]

Next let Pₕ=−μgh where ρ is blood density, g is gravitational acceleration, and h is the height of the distal measurement site relative to the reference point (e.g., the heart), with the upward direction treated as positive (i.e., h>0 indicates a position above the heart). Substituting for Pₕ:

c=k₁·e^{k}^{₂}^{(P-ρgh)}. [39]

To find PTT, first substitute in the differential definition of c:

$$\frac{d x}{d t} = {k}_{1} \cdot {e}^{{k}_{2} ( P - ρ g h )} , \qquad [ 40 ]$$

In the above, x is the position of the wave along the arm and t is time. Rearrange then integrate to yield PTT,

$$P T T = {\int }_{0}^{L} \frac{d x}{{k}_{1} \cdot {e}^{{k}_{2} ( P - ρ g h )}} . \qquad [ 41 ]$$

Note that h is a function of the distance the wave has traveled. Thus, h can be redefined in terms of x. To do so, parameterize the arm as two rigid bodies (upper arm and forearm) of lengths Lᵤ and L_{ƒ} Next, let the point of reference for relative altitude be the shoulder (i.e., x=0) such that the relative altitude for any point along the arm can be defined as follows:

$$h = \{ \begin{matrix}x sin {θ}_{u} , & 0 \leq x \leq {L}_{u} \\ {L}_{u} sin {θ}_{u} + ( x - {L}_{u} ) sin {θ}_{f} , & {L}_{u} \leq x \leq {L}_{f} ,\end{matrix} \qquad [ 42 ]$$

In the above, θᵤ is the upper arm pitch and θ_{ƒ} is the forearm pitch, with positive pitch indicating moving the limb upward. Next, split the integral into upper arm and forearm components, as follows:

$$PTT = {\int }_{0}^{{L}_{u}} \frac{d x}{{k}_{1} \cdot exp [ {k}_{2} ( P - x \cdot ρ g sin {θ}_{u} ) ]} + {\int }_{{L}_{u}}^{{L}_{f}} \frac{d x}{{k}_{1} \cdot exp [ {k}_{2} ( P - {L}_{u} \cdot ρ g sin {θ}_{u} - [ x - {L}_{u} ] \cdot ρ g sin {θ}_{f} ) ]} \qquad [ 43 ]$$

If θᵤ and θ_{ƒ} are assumed constant for a cardiac cycle, Eqn. 43 can be directly integrated, yielding Eqn. 31, the model for PTT with Pₕ effects compensated using arm pose. Rearranging this equation to solve for P yields Eqn. 32.

PTT Simulation. PTT was simulated with Eqn. 31 using MATLAB. For all simulations, the parameters were k₁=80 cm·s⁻¹, k₂=0.0165 mmHg⁻¹, P=90 mmHg, and L=60 cm with Lᵤ=L_{ƒ}=½ L, unless otherwise noted. For simulations showing the PTT-BP relationship, BP was varied from 50 to 200 mmHg in increments of 5 mmHg. For simulations showing the relationship between PTT and arm pose, θᵤ and θ_{ƒ} were varied from −90 to 90° in 5° increments.

PTT, pose, and BP data preprocessing. Raw PPG recordings were filtered using a 2^{nd} order low pass Butterworth filter with a cutoff frequency of 15 Hz. To minimize the effects of movement artifacts, filtered PPG data were cleaned using the 7-step pulse wave filter (56). Additionally, 5 seconds of data between h transitions were dropped, to remove data from known periods of motion. Onset times were identified as the peaks in the twice-differentiated signal that occurred prior to a PPG peak. ECG data were used without additional cleaning. The R-wave peaks were identified as the proximal pulse reference. PTT was calculated as the time difference between a PPG onset and the preceding R-wave peak.

An embodiment of the deep learning model was used to generate arm pose estimates for each participant using the preprocessed IMU data. The arm pose predictions were upsampled to 2000 Hz. For each PTT, the arm pose corresponding to the R-wave peak and PPG onset were averaged and used to approximate arm pose for that cardiac cycle.

Raw BP signals were filtered using a 2^{nd} order low pass Butterworth filter with a cutoff frequency of 30 Hz. DBP and SBP were identified as the minimum and maximum of each beat, respectively. Mean arterial pressure was calculated as ⅔ ·DBP+⅓·SBP, a commonly used approximation (57). BP was matched to the PTT calculated from the simultaneously acquired PPG beat.

Best-fit PTT prediction. For each participant, data corresponding to the initial rest period and the first three movements (i.e., the first ˜8 minutes of data) were used for calibration, with the remaining eight movement stages held out for testing. The first four minutes of data from the rest stage was discarded, as the initial minutes were excessively noisy for many participants. The person-specific coefficients, k₁ and k₂, were found by minimizing the mean squared error between measured and predicted PTT with mean arterial pressure as P. During calibration of the “uncorrected” baseline model, θᵤ and θ_{ƒ} were constrained to 0° (i.e., αᵤ and α_{ƒ} set to 1). For the “corrected” model, θᵤ and θ_{ƒ} were the predicted and measured arm pitch, respectively. After calibration, best-fit PTT predictions were generated with Eqn. 31 using the same data used for calibration. During PTT prediction, the uncorrected baseline model constrained pitch to 0°.

BP prediction. The person-specific coefficients were found by minimizing the mean squared error of measured and predicted BP on the data previously used for PTT model calibration. For DBP prediction, the model was calibrated using DBP as P. Conversely, the model was calibrated with SBP as P for SBP prediction. After calibration, BP predictions were generated with Eqn. 32 on the testing data for each participant. During prediction with the uncorrected model, both θᵤ and θ_{ƒ} were constrained to 0°.

Statistical analysis. For statistical analysis between two groups with matched samples, a paired two-tailed Student's t-test was used. For comparison between multiple groups of matched samples with one variable, a mixed-effects model with Dunnet post-hoc test for multiple-comparisons was used. For analyzing multiple groups of matched samples with two variables, a mixed-effects model with Šidák post-hoc test for multiple-comparisons was used. For analysis of repeated measures correlation coefficients, the Williams' test for comparing two dependent correlations sharing one variable was used. Significance was considered for P<0.05. All data were expressed as the mean±standard error of the mean unless otherwise indicated. Statistical tests were calculated in GraphPad Prism 9.0.

Acknowledgments. This work was supported in part by grants from the National Institutes of Health (UL1 TR001873), the National Science Foundation (Graduate Research Fellowship under Grant No. DGE 1644869), and the Columbia University Biomedical Engineering Technology Accelerator.

FIG. 25 illustrates an overview of approach for tracking arm orientation. Panel A depicts an overview of approach for Pₕ tracking. Embodiments use wrist-based inertial sensors and a deep learning model to infer arm orientation, which is then used to calculate Pₕ to correct errors that result from height differences between BP sensors using an analytical biomechanics wave model. Panel B depicts a diagram illustrating changes in Pₕ along the arm relative to the heart. The arrows represent the pulse wave traveling down the arm, with arrow length corresponding to PWV magnitude. Panel C depicts PTT calculation from ECG and PPG waveforms. Panel D depicts a block diagram of an example deep learning-assisted model and BP prediction pipeline. Arm pose is estimated from a wrist-based IMU and a deep learning model based on measurements from IMU and a parametrized arm-pose coordinate system; this arm pose information is used to calculate hydrostatic pressure (Pₕ). PTT is measured using ECG and PPG. A prediction of BP is made using an analytical pressure wave propagation model with inputs of PTT and Pₕ following person-specific calibration. Panel E depicts device use in an example implementation, include one lead ECG, finger PPG, and wrist-mounted IMU.

FIG. 26 illustrates techniques for tracking of arm pose from a single wrist-based IMU using parametrized arm-pose coordinate system and deep learning. Panel A depicts a schematic diagram showing a parameterized model for arm pose. In this example, positive θ indicates moving the corresponding limb upward. Panel B depicts an example deep learning architecture diagram for tracking upper arm orientation. In some embodiments, the inputs at each timestep can be forearm acceleration and orientation (e.g., yaw, pitch, and roll) represented as a unit quaternion. These inputs can be fed through a fully connected (FC) layer followed by two bidirectional LSTM (BiLSTM) layers. The latent feature vector for the current timestep can then passed through a final FC layer to predict the upper arm orientation quaternion, normalized to unit norm. The orientation quaternion is finally used to calculate θᵤ. Panel C depicts a histogram of absolute errors for θᵤ prediction for an implemented embodiment of the model pre-trained on the Virginia Tech Natural Motion Dataset alone (“pre-trained”) and after fine-tuning on in-house training data (“fine-tuned”). Panel D depicts a time series of predicted and measured θᵤ for a representative participant from a test fold.

FIG. 27 illustrates diagrams that depict changes in PTT induced by hydrostatic pressure, as modeled analytically and experimentally validated. Panel A depicts a relationship between pressure and simulated PTT. The solid black line indicates simulated PTT without Pₕ effects, and the dashed blue and orange lines indicate simulated PTT with Pₕ minimized and maximized via straight up and down poses, respectively. Panel B Left depicts a heatmap of simulated PTTs for constant BP over possible arm pose configurations. Positive pitch indicates moving the corresponding limb upward. Panel B Left depicts a projection of a heatmap simulation with arm pose converted to hand height. Solid line indicates simulated PTT with average Pₕ effects (i.e., where θᵤ=θ_{ƒ}). Dashed blue and orange lines indicate simulated PTT with Pₕ minimized and maximized, respectively. Panel C depicts box plots of measured PTT taken at different hand heights. Each measurement was the participant's time-averaged PTT for the indicated height (for h=−25, 0, and 25 cm, n=20, 20, and 19, respectively. Significance determined by mixed-effects model followed by Dunnett post-hoc test. For h=−25 vs h=0 cm, ****P<0.0001; for h=0 vs h=25 cm, ****P=0.00001). Individual trajectories illustrated in FIG. 32. Panel B depicts a time series of measured and predicted hand height (top); corresponding time series of PTT predicted using the uncorrected and corrected models compared to the measured reference (bottom). Time series of PTT prediction error illustrated in FIG. 33. Panel E depicts box plots of h-stratified MAE for best-fit PTT predictions generated using the uncorrected and corrected model compared to the measured reference. Each measurement was the participant's time-averaged MAE for the indicated h.

The diagrams of FIG. 27 show an illustrative example of arm pose corresponding to each group (for h=−25, 0, and 25 cm, n=16, 20, and 15, respectively. Significance determined by mixed-effects model followed by Šidák post-hoc test. For h=−25 cm, **P=0.0005; for h=0 cm, P=0.0708; for h=25 cm, **P=0.0012). Individual trajectories are illustrated in FIG. 35. Panel F depicts repeated measures Bland-Altman plots for the best-fit PTT predictions from the uncorrected (top) and corrected model (bottom) compared to the measured reference. X-axis shows the average of prediction and reference, and the y-axis shows the difference between prediction and reference. Points correspond to time-averaged participant-h pairs, not individual participants. Solid line indicates the mean difference, and the dashed line indicates 95% LoA (I=51; 2 to 3 measurements from each participant). Enlarged version showing the values of h illustrated in FIG. 36. For panels C and E, the box represents the interquartile range, with the horizontal line at the median value. The vertical lines extend to the maximum and minimum data points within 1.5×IQR.

FIG. 28 illustrates diagrams of BP with correction for hydrostatic pressure error. Panel A depicts time series of measured and predicted hand height (top); corresponding time series of DBP and SBP predicted using the uncorrected and corrected models compared to the measured reference (bottom). Time series of BP prediction error illustrated in FIG. 38. Panel B depicts box plots of h-stratified MAE for DBP (middle) and SBP (bottom) predictions generated using the uncorrected and corrected model compared to the measured reference. Each measurement was the participant's time-averaged MAE for the indicated h. The box represents the interquartile range, with the horizontal line at the median value. The vertical lines extend to the maximum and minimum data points within 1.5×IQR. The diagrams (top) show an illustrative example of arm pose corresponding to each group (for h=−25, 0, and 25 cm, n=19, 20, and 19, respectively. Significance determined by mixed-effects model followed by Šidák post-hoc test. DBP: for h=−25 cm, ****P<0.0001; for h=0 cm, *P=0.0356; for h=25 cm, **P=0.0096. SBP: for h=−25 cm, ****P<0.0001; for h=0 cm, *P=0.0284; for h=25 cm, **P=0.0051). Individual trajectories illustrated in FIGS. 41 and 42. Panels C and D depict repeated measures Bland-Altman plots using the uncorrected and corrected model compared to the measured reference for DBP (Panel C) and SBP (Panel D) prediction. X-axis shows the average of prediction and reference, and the y-axis shows the difference between prediction and reference. Points correspond to time-averaged participant-h pairs, not individual participants. Solid line indicates the mean difference, and the dashed line indicates 95% LoA (n=58; 2 to 3 measurements from each participant). Enlarged versions showing the values of h illustrated in FIGS. 43 and 44.

TABLE 1
Summary of model performance on best-fit PTT prediction.

| | PTT (ms) | |
|---|---|---|
| | Uncorrected | Corrected |
| Overall | 10.6 ± 1.2 | 6.4 ± 0.5**** |
| h = −25 cm | 14.3 ± 2.4 | 6.7 ± 1.1** |
| h = 0 cm | 7.0 ± 0.6 | 6.2 ± 0.5 |
| h = 25 cm | 26.0 ± 4.1 | 8.8 ± 3.0*** |

MAE of best-fit PTT predictions using the uncorrected and corrected models aggregated over participants (for overall, n = 20. Significance determined by paired t-test; for h = −25, 0, and 25 cm, n = 16, 20, and 15, respectively. Significance determined by mixed-effects model followed by \check{S}ídák post-hoc test. For overall, ****P < 0.0001; for h = −25 cm, **P = 0.0005; for h = 0 cm, P = 0.0708; for h = 25 cm, **P = 0.0012). Each measurement was the participant's time-averaged MAE overall or for the indicated h. Data is represented as mean ± standard error of the mean.

TABLE 2
Summary of model performance for BP estimation.

| | DBP (mmHg) | | SBP (mmHg) | |
|---|---|---|---|---|
| | Uncorrected | Corrected | Uncorrected | Corrected |
| Overall | 10.6 ± 0.6 | 6.8 ± 0.4**** | 9.6 ± 0.6 | 5.9 ± 0.5**** |
| h = −25 | 15.0 ± 1.0 | 6.8 ± 0.5**** | 13.5 ± 1.1 | 5.9 ± 0.7**** |
| cm | | | | |
| h = 0 cm | 7.3 ± 0.5 | 6.5 ± 0.5* | 6.6 ± 0.6 | 5.8 ± 0.6* |
| h = 25 cm | 11.5 ± 1.5 | 7.8 ± 0.8** | 9.6 ± 1.1 | 5.9 ± 0.5** |

MAE of DBP and SBP prediction using the uncorrected and corrected models aggregated over participants (for overall, n = 20. Significance determined by paired t-test; for h = −25, 0, and 25 cm, n = 19, 20, and 19, respectively. Significance determined by mixed-effects model followed by \check{S}ídák post-hoc test. DBP: for overall, ****P < 0.0001; for h = −25 cm, ****P < 0.0001; for h = 0 cm, *P = 0.0356; for h = 25 cm, **P = 0.0096. SBP: for overall, ****P < 0.0001; for h = −25 cm, ****P < 0.0001; for h = 0 cm, *P = 0.0284; for h = 25 cm, **P = 0.0051). Each measurement was the participant's time-averaged MAE overall or for the indicated h. Data is represented as mean ± standard error of the mean.

FIG. 29 illustrates diagrams for intrinsic ZYX Tait-Bryan angles and IMU alignment. Panel A depicts intrinsic ZYX Tait-Bryan angles as measured by an IMU in the North-West-Up coordinate system. Roll represents rotation about the intrinsic (sensor-frame) X-axis. Pitch represents rotation around the intrinsic Y-axis. Yaw represents rotation along the intrinsic Z-axis. Panel B depicts a top-down view of the arm showing IMU placement on the wrist. The 3D axes indicate the alignment of the sensor with the arm. Panel C depicts a side view of the arm. Pitch is equal to the angle the forearm makes with the horizontal plane, θ_{ƒ}.

FIG. 30 illustrates graphs that depict a dependence of PTT on pressure for varied parameters, in an analytical wave-propagation equation. Panels A and C depict simulations with Eqn. 31 showing the impact of model parameters on the PTT-pressure relationship with θᵤ=θ_{ƒ}=0°. Default model parameters k₁=80 cm·s⁻¹, k₂=0.0165 mmHg⁻¹, and L=60 cm with Lᵤ=L_{ƒ}=½ L. (Panel A) k₁ varied between 40, 80, 120 cm·s⁻¹. (Panel B) k₂ varied between 0.0115, 0.0165, and 0.0225 mmHg⁻¹. (Panel C) L varied between 40, 60, and 80 cm.

FIG. 31 illustrates graphs that depict a dependence of PTT on h, in an analytical wave-propagation equation. Panels A and C depict simulations with Eqn. 31 showing the impact of model parameters on the PTT-h relationship. Default model parameters k₁=80 cm·s⁻¹, k₂=0.0165 mmHg⁻¹, P=90 mmHg, and L=60 cm with Lᵤ=L_{ƒ}=½ L. Solid line indicates where θᵤ=θ_{ƒ} Dashed line indicates the bounds of possible PTT predictions. (Panel A) k₁ varied between 40, 80, 120 cm·s⁻¹. (Panel B) k₂ varied between 0.0115, 0.0165, and 0.0225 mmHg⁻¹. (Panel C) L varied between 40, 60, and 80 cm.

FIG. 32 illustrates a PTT vs h estimation plot, based on data from human subjects. The left graph depicts individual trajectories of PTT taken at different hand heights. Each point shows the participant's time-averaged PTT for the indicated height with lines connecting data from the same participant (for h=−25, 0, and 25 cm, n=20, 20, and 19, respectively. Significance determined by mixed-effects model followed by Dunnett post-hoc test. For h=−25 vs h=0 cm,****P<0.0001; for h=0 vs h=25 cm, ****P=0.00001). The right graph depicts a change in PTT for each participant following a decrease or increase in h compared h=0 cm. The center line shows the mean, and the error bars shows the 95% confidence interval (for −25−0, n=20; for 25−0, n=19).

FIG. 33 illustrates a time series of best-fit PTT prediction error for the uncorrected and corrected models compared to the measured reference from a representative participant.

FIG. 34 illustrates an estimation plot for best-fit PTT prediction MAE. The left plot depicts MAE for best-fit PTT predictions for individual participants using the uncorrected and corrected models compared to the measured reference. The box plots show the distribution in MAE for the uncorrected and corrected models. The box represents the interquartile range, with the horizontal line at the median value. The vertical lines extend to the maximum and minimum data points within 1.5×IQR. Each point between the box plots shows individual participant MAE for the entire calibration interval with lines connecting data from the same participant (n=20. Significance determined by paired t-test. ****P<0.0001). The right plot depicts a difference in MAE between the two models for each participant. The center line shows the mean, and the error bars shows the 95% confidence interval (n=20).

FIG. 35 illustrates an estimation plot for best-fit PTT prediction MAE stratified by h. The left plot depicts MAE for best-fit PTT predictions for individual participants using the uncorrected and corrected models compared to the measured reference stratified by h. Each point shows the participant's MAE for the indicated height with lines connecting data from the same participant (for h=−25, 0, and 25 cm, n=16, 20, and 15, respectively. Significance determined by mixed-effects model followed by Šidák post-hoc test. For h=−25 cm, **P=0.0005; for h=0 cm, P=0.0708; for h=25 cm, **P=0.0012). The right plot depicts a difference in MAE between the two models for each participant at the indicated height. The center line shows the mean, and the error bars shows the 95% confidence interval (for h=−25, 0, and 25 cm, n=16, 20, and 15, respectively).

FIG. 36 illustrates PTT prediction Bland-Altman plots. Panels A and B depict repeated measures Bland-Altman plots for the best-fit PTT predictions from the uncorrected (Panel A) and corrected model (Panel B) compared to the measured reference. X-axis shows the average of prediction and reference, and the y-axis shows the difference between prediction and reference. Points correspond to time-averaged participant-h pairs, not individual participants. Solid line indicates the mean difference, and the dashed line indicates 95% LoA (n=51; 2 to 3 measurements from each participant).

FIG. 37 illustrates repeated measures correlation for best-fit PTT prediction plots. Panels A and B depict repeated measures correlation plot for best-fit PTT predictions from the uncorrected (Panel A) and corrected (Panel B) models compared to the measured reference over the entire calibration interval. This representation is based on the dataset shown in FIG. 27 panels E and F. Points correspond to time-averaged participant-h pairs, not individual participants (n=51; 2 to 3 measurements from each participant).

FIG. 38 illustrates time series BP prediction error plots. Panels A and B depict time series of DBP (Panel A) and SBP (Panel B) prediction error for the uncorrected and corrected models compared to the measured reference from a representative participant.

FIG. 39 illustrates estimation plots for DBP prediction MAE. The left plot depicts MAE for DBP predictions for individual participants using the uncorrected and corrected models compared to the measured reference. The box plots show the distribution in MAE for the uncorrected and corrected models. The box represents the interquartile range, with the horizontal line at the median value. The vertical lines extend to the maximum and minimum data points within 1.5×IQR. Each point between the box plots shows individual participant MAE for the entire testing interval with lines connecting data from the same participant (n=20. Significance determined by paired t-test. ****P<0.0001). The right plot depicts a difference in MAE between the two models for each participant. The center line shows the mean, and the error bars shows the 95% confidence interval (n=20).

FIG. 40 illustrates estimation plots for SBP prediction MAE. The left plot depicts MAE for SBP predictions for individual participants using the uncorrected and corrected models compared to the measured reference. The box plots show the distribution in MAE for the uncorrected and corrected models. The box represents the interquartile range, with the horizontal line at the median value. The vertical lines extend to the maximum and minimum data points within 1.5×IQR. Each point between the box plots shows individual participant MAE for the entire testing interval with lines connecting data from the same participant (n=20. Significance determined by paired t-test. ****P<0.0001). The right plot depicts a difference in MAE between the two models for each participant. The center line shows the mean, and the error bars shows the 95% confidence interval (n=20).

FIG. 41 illustrates estimation plots for DBP prediction MAE stratified by h. The left plot depicts MAE for DBP predictions from individual participants using the uncorrected and corrected models compared to the measured reference stratified by h. Each point shows the participant's MAE for the indicated height with lines connecting data from the same participant (for h=−25, 0, and 25 cm, n=19, 20, and 19, respectively. Significance determined by mixed-effects model followed by Šidák post-hoc test. For h=−25 cm, ****P<0.0001; for h=0 cm, *P=0.0356; for h=25 cm, **P=0.0096.). The right plot depicts a difference in MAE between the two models for each participant at the indicated height. The center line shows the mean, and the error bars shows the 95% confidence interval (for h=−25, 0, and 25 cm, n=19, 20, and 19, respectively).

FIG. 42 illustrates estimation plots for SBP prediction MAE stratified by h. The left plot depicts MAE for SBP predictions from individual participants using the uncorrected and corrected models compared to the measured reference stratified by h. Each point shows the participant's MAE for the indicated height with lines connecting data from the same participant (for h=−25, 0, and 25 cm, n=19, 20, and 19, respectively. Significance determined by mixed-effects model followed by Šidák post-hoc test. For h=−25 cm, ****P<0.0001; for h=0 cm, *P=0.0284; for h=25 cm, **P=0.0051). The right plot depicts a difference in MAE between the two models for each participant at the indicated height. The center line shows the mean, and the error bars shows the 95% confidence interval (for h=−25, 0, and 25 cm, n=19, 20, and 19, respectively).

FIG. 43 illustrates prediction Bland-Altman plots, with data from three heights shown separately. Panels A and B depict repeated measures Bland-Altman plots for DBP prediction using the uncorrected (Panel A) and corrected model (Panel B) compared to the measured reference. X-axis shows the average of prediction and reference, and the y-axis shows the difference between prediction and reference. Points correspond to time-averaged participant-h pairs, not individual participants. Solid line indicates the mean difference, and the dashed line indicates 95% LoA (n=58; 2 to 3 measurements from each participant).

FIG. 44 illustrates SBP prediction Bland-Altman plots, with data from three heights shown separately. Panels A and B depict repeated measures Bland-Altman plots for SBP prediction using the uncorrected (Panel A) and corrected model (Panel B) compared to the measured reference. X-axis shows the average of prediction and reference, and the y-axis shows the difference between prediction and reference. Points correspond to time-averaged participant-h pairs, not individual participants. Solid line indicates the mean difference, and the dashed line indicates 95% LoA (n=58; 2 to 3 measurements from each participant).

TABLE S1
Summary statistics for study participants.

| | n = 20 all | n = 5 males | n = 15 females |
|---|---|---|---|
| Age (years) | 28.4 ± 6.0 | 33.2 ± 7.9 | 26.7 ± 4.1 |
| Weight (kg) | 70.7 ± 16.8 | 81.3 ± 18.7 | 67.2 ± 14.5 |
| Height (cm) | 170.4 ± 23.9 | 176.1 ± 6.1 | 168.5 ± 27.1 |
| BMI | 25.1 ± 6.7 | 26.1 ± 5.1 | 24.7 ± 7.1 |
| Arm Length (cm) | 50.4 ± 5.9 | 53.7 ± 3.2 | 49.3 ± 6.2 |
| Hand Length | 18.5 ± 1.4 | 20.1 ± 1.0 | 18.0 ± 1.0 |
| (cm) | | | |
| SBP (mmHg) | 116.1 ± 9.3 | 113.8 ± 5.0 | 116.9 ± 10.2 |
| DBP (mmHg) | 74.7 ± 6.0 | 71.0 ± 1.8 | 75.9 ± 6.4 |

Summary of intake data collected from the 20 study participants. Data is represented as mean±standard deviation.

Embodiments can enable non-invasive monitoring of BP (e.g., ambulatory monitoring and/or long-term monitoring throughout a patient's day). For example, a patient that has been diagnosed with a BP condition, such as hypertension, may be prescribed treatments for the condition, and the non-invasive monitoring can be used to assess treatment success. At times, the treatment can be one or more drug prescriptions, and the non-invasive BP monitoring can be used to obtain a high fidelity view of the treatment outcome (e.g., the effect on the patient's BP). The non-invasive BP monitoring enabled by embodiments can be used for either in-patient (in facility) or out-patient (at home) treatments. In another example, non-invasive BP monitoring can be used by the general population, such as through implementation in a standard wearable device. For example, the general population can learn more about how BP impacts anxiety, stress, overall health, and other quality of life factors, and to support preventive measures.

It will be appreciated that the modules, processes, systems, and sections described above can be implemented in hardware, hardware programmed by software, software instruction stored on a non-transitory computer readable medium or a combination of the above. For example, a method for measuring blood pressure can be implemented, for example, using a processor configured to execute a sequence of programmed instructions stored on a non-transitory computer readable medium. For example, the processor can include, but not be limited to, a personal computer or workstation or other such computing system that includes a processor, microprocessor, microcontroller device, or is comprised of control logic including integrated circuits such as, for example, an Application Specific Integrated Circuit (ASIC). The instructions can be compiled from source code instructions provided in accordance with a programming language such as Java, C++, C#.net or the like. The instructions can also comprise code and data objects provided in accordance with, for example, the Visual Basic™ language, Lab VIEW, or another structured or object-oriented programming language. The sequence of programmed instructions and data associated therewith can be stored in a non-transitory computer-readable medium such as a computer memory or storage device which may be any suitable memory apparatus, such as, but not limited to read-only memory (ROM), programmable read-only memory (PROM), electrically erasable programmable read-only memory (EEPROM), random-access memory (RAM), flash memory, disk drive and the like.

Furthermore, the modules, processes, systems, and sections can be implemented as a single processor or as a distributed processor. Further, it should be appreciated that the steps mentioned above may be performed on a single or distributed processor (single and/or multi-core). Also, the processes, modules, and sub-modules described in the various figures of and for embodiments above may be distributed across multiple computers or systems or may be co-located in a single processor or system. Exemplary structural embodiment alternatives suitable for implementing the modules, sections, systems, means, or processes described herein are provided below.

The modules, processors or systems described above can be implemented as a programmed general purpose computer, an electronic device programmed with microcode, a hard-wired analog logic circuit, software stored on a computer-readable medium or signal, an optical computing device, a networked system of electronic and/or optical devices, a special purpose computing device, an integrated circuit device, a semiconductor chip, and a software module or object stored on a computer-readable medium or signal, for example.

Embodiments of the method and system (or their sub-components or modules), may be implemented on a general-purpose computer, a special-purpose computer, a programmed microprocessor or microcontroller and peripheral integrated circuit element, an ASIC or other integrated circuit, a digital signal processor, a hardwired electronic or logic circuit such as a discrete element circuit, a programmed logic circuit such as a programmable logic device (PLD), programmable logic array (PLA), field-programmable gate array (FPGA), programmable array logic (PAL) device, or the like. In general, any process capable of implementing the functions or steps described herein can be used to implement embodiments of the method, system, or a computer program product (software program stored on a non-transitory computer readable medium).

Furthermore, embodiments of the disclosed method, system, and computer program product may be readily implemented, fully or partially, in software using, for example, object or object-oriented software development environments that provide portable source code that can be used on a variety of computer platforms. Alternatively, embodiments of the disclosed method, system, and computer program product can be implemented partially or fully in hardware using, for example, standard logic circuits or a very-large-scale integration (VLSI) design. Other hardware or software can be used to implement embodiments depending on the speed and/or efficiency requirements of the systems, the particular function, and/or particular software or hardware system, microprocessor, or microcomputer being utilized. Embodiments of the method, system, and computer program product can be implemented in hardware and/or software using any known or later developed systems or structures, devices and/or software by those of ordinary skill in the applicable art from the function description provided herein and with a general basic knowledge of blood pressure measurement and/or computer programming arts.

Moreover, embodiments of the disclosed method, system, and computer program product can be implemented in software executed on a programmed general purpose computer, a special purpose computer, a microprocessor, or the like.

It is, thus, apparent that there is provided, in accordance with the present disclosure, blood pressure measurement devices, methods, and systems. Many alternatives, modifications, and variations are enabled by the present disclosure. Features of the disclosed embodiments can be combined, rearranged, omitted, etc., within the scope of the invention to produce additional embodiments. Furthermore, certain features may sometimes be used to advantage without a corresponding use of other features. Accordingly, Applicants intend to embrace all such alternatives, modifications, equivalents, and variations that are within the spirit and scope of the present invention.

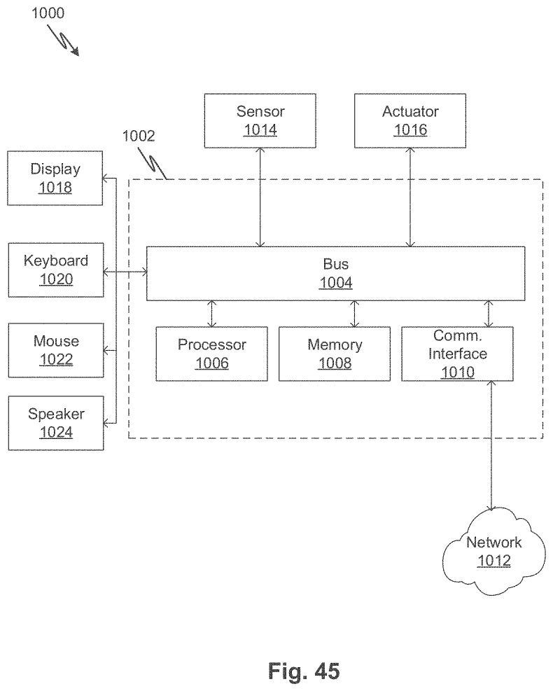

FIG. 45 shows a block diagram of an example computer system according to embodiments of the disclosed subject matter. In various embodiments, all or parts of system 1000 may be embedded in a system such as a diagnostic device. In these embodiments, all or parts of system 1000 may provide the functionality of a controller of the medical treatment device/systems. In some embodiments, all or parts of system 1000 may be implemented as a distributed system, for example, as a cloud-based system.

System 1000 includes a computer 1002 such as a personal computer or workstation or other such computing system that includes a processor 1006. However, alternative embodiments may implement more than one processor and/or one or more microprocessors, microcontroller devices, or control logic including integrated circuits such as ASIC.

Computer 1002 further includes a bus 1004 that provides communication functionality among various modules of computer 1002. For example, bus 1004 may allow for communicating information/data between processor 1006 and a memory 1008 of computer 1002 so that processor 1006 may retrieve stored data from memory 1008 and/or execute instructions stored on memory 1008. In one embodiment, such instructions may be compiled from source code/objects provided in accordance with a programming language such as Java, C++, C#, .net, Visual Basic™ language, LabVIEW, or another structured or object-oriented programming language. In one embodiment, the instructions include software modules that, when executed by processor 1006, provide renal replacement therapy functionality according to any of the embodiments disclosed herein.

Memory 1008 may include any volatile or non-volatile computer-readable memory that can be read by computer 1002. For example, memory 1008 may include a non-transitory computer-readable medium such as ROM, PROM, EEPROM, RAM, flash memory, disk drive, etc. Memory 1008 may be a removable or non-removable medium.

Bus 1004 may further allow for communication between computer 1002 and a display 1018, a keyboard 1020, a mouse 1022, and a speaker 1024, each providing respective functionality in accordance with various embodiments disclosed herein, for example, for configuring a treatment for a patient and monitoring a patient during a treatment.

Computer 1002 may also implement a communication interface 1010 to communicate with a network 1012 to provide any functionality disclosed herein, for example, for alerting a healthcare professional and/or receiving instructions from a healthcare professional, reporting patient/device conditions in a distributed system for training a machine learning algorithm, logging data to a remote repository, etc. Communication interface 1010 may be any such interface known in the art to provide wireless and/or wired communication, such as a network card or a modem.

Bus 1004 may further allow for communication with a sensor 1014 and/or an actuator 1016, each providing respective functionality in accordance with various embodiments disclosed herein, for example, for measuring signals indicative of a patient/device condition and for controlling the operation of the device accordingly. For example, sensor 1014 may provide a signal indicative of a viscosity of a fluid in a fluid circuit in a renal replacement therapy device, and actuator 1016 may operate a pump that controls the flow of the fluid responsively to the signals of sensor 1014.

### REFERENCES

1. B. M. Psaty et al., Association between blood pressure level and the risk of myocardial infarction, stroke, and total mortality—The cardiovascular health study. Arch Intern Med 161, 1183-1192 (2001).
2. W. B. Kannel, Blood pressure as a cardiovascular risk factor—Prevention and treatment. Jama-J Am Med Assoc 275, 1571-1576 (1996).
3. S. Lewington et al., Age-specific relevance of usual blood pressure to vascular mortality: a meta-analysis of individual data for one million adults in 61 prospective studies. Lancet 360, 1903-1913 (2002).
4. P. K. Whelton et al., 2017 ACC/AHA/AAPA/ABC/ACPM/AGS/APhA/ASH/ASPC/NMA/PCNA Guideline for the Prevention, Detection, Evaluation, and Management of High Blood Pressure in Adults: Executive Summary: A Report of the American College of Cardiology/American Heart Association Task Force on Clinical Practice Guidelines. Circulation 138, e426-e483 (2018).
5. T. Unger et al., 2020 International Society of Hypertension Global Hypertension Practice Guidelines. Hypertension 75, 1334-1357 (2020).
6. M. Walsh et al., Relationship between Intraoperative Mean Arterial Pressure and Clinical Outcomes after Noncardiac Surgery: Toward an Empirical Definition of Hypotension. Anesthesiology 119, 507-515 (2013).
7. H. Lahrmann et al., EFNS guidelines on the diagnosis and management of orthostatic hypotension. Eur J Neurol 13, 930-936 (2006).
8. J. Kooman et al., EBPG guideline on haemodynamic instability. Nephrol Dial Transplant 22 Suppl 2, ii22-44 (2007).
9. A. L. Siu, U. S. P. S. T. Force, Screening for high blood pressure in adults: U.S. Preventive Services Task Force recommendation statement. Ann Intern Med 163, 778-786 (2015).
10. P. Muntner et al., Measurement of Blood Pressure in Humans: A Scientific Statement From the American Heart Association. Hypertension 73, e35-e66 (2019).
11. M. Gorostidi, E. Vinyoles, J. R. Banegas, A. de la Sierra, Prevalence of white-coat and masked hypertension in national and international registries. Hypertens Res 38, 1-7 (2015).
12. D. E. Anstey et al., Diagnosing Masked Hypertension Using Ambulatory Blood Pressure Monitoring, Home Blood Pressure Monitoring, or Both? Hypertension 72, 1200-1207 (2018).
13. E. Grossman, Ambulatory blood pressure monitoring in the diagnosis and management of hypertension. Diabetes Care 36 Suppl 2, S307-311 (2013).
14. A. J. Viera, K. Lingley, A. L. Hinderliter, Tolerability of the Oscar 2 ambulatory blood pressure monitor among research participants: a cross-sectional repeated measures study. Bmc Med Res Methodol 11 (2011).
15. R. Agarwal, R. P. Light, The Effect of Measuring Ambulatory Blood Pressure on Nighttime Sleep and Daytime Activity-Implications for Dipping. Clin J Am Soc Nephro 5, 281-285 (2010).
16. A. Chandrasekhar et al., Smartphone-based blood pressure monitoring via the oscillometric finger-pressing method. Sci Transl Med 10 (2018).
17. J. R. Sempionatto et al., An epidermal patch for the simultaneous monitoring of haemodynamic and metabolic biomarkers. Nat Biomed Eng 10.1038/s41551-021-00685-1 (2021).
18. N. Q. Luo et al., Flexible Piezoresistive Sensor Patch Enabling Ultralow Power Cuffless Blood Pressure Measurement. Advanced Functional Materials 26, 1178-1187 (2016).
19. K. Y. Meng et al., Flexible Weaving Constructed Self-Powered Pressure Sensor Enabling Continuous Diagnosis of Cardiovascular Disease and Measurement of Cuffless Blood Pressure. Advanced Functional Materials 29 (2019).
20. C. H. Wang et al., Monitoring of the central blood pressure waveform via a conformal ultrasonic device. Nat Biomed Eng 2, 687-695 (2018).
21. P. L. Mitchell, R. W. Parlin, H. Blackburn, Effect of Vertical Displacement of the Arm on Indirect Blood-Pressure Measurement. N Engl J Med 271, 72-74 (1964).
22. B. P. Imholz et al., Feasibility of ambulatory, continuous 24-hour finger arterial pressure recording. Hypertension 21, 65-73 (1993).
23. A. Filippeschi et al., Survey of Motion Tracking Methods Based on Inertial Sensors: A Focus on Upper Limb Human Motion. Sensors (Basel) 17 (2017).
24. O. Woodman, An introduction to inertial navigation (Technical Report No. 696). Cambridge, UK: University of Cambridge Computer Laboratory (2007).
25. Y. B. Son, S. Oh, A Barometer-IMU Fusion Method for Vertical Velocity and Height Estimation. Ieee Sensor, 402-405 (2015).
26. A. M. Sabatini, V. Genovese, A Sensor Fusion Method for Tracking Vertical Velocity and Height Based on Inertial and Barometric Altimeter Measurements. Sensors (Basel) 14, 13324-13347 (2014).
27. A. Manivannan, W. C. B. Chin, A. Barrat, R. Bouffanais, On the Challenges and Potential of Using Barometric Sensors to Track Human Activity. Sensors (Basel) 20 (2020).
28. C. C. Y. Poon, Y. T. Zhang, Using the changes in hydrostatic pressure and pulse transit time to measure arterial blood pressure. 2007 Annual International Conference of the Ieee Engineering in Medicine and Biology Society, Vols 1-16 Doi 10.1109/Iembs.2007.4352794, 2336-2337 (2007).
29. J. Lee, S. Yang, S. Lee, H. C. Kim, Analysis of Pulse Arrival Time as an Indicator of Blood Pressure in a Large Surgical Biosignal Database: Recommendations for Developing Ubiquitous Blood Pressure Monitoring Methods. J Clin Med 8 (2019).
30. R. Mukkamala et al., Toward Ubiquitous Blood Pressure Monitoring via Pulse Transit Time: Theory and Practice. IEEE Trans Biomed Eng 62, 1879-1901 (2015).
31. G. Pucci et al., Age-Specific Acute Changes in Carotid-Femoral Pulse Wave Velocity With Head-up Tilt. Am J Hypertens 33, 1112-1118 (2020).
32. A. Tripathi et al., A Pulse Wave Velocity Based Method to Assess the Mean Arterial Blood Pressure Limits of Autoregulation in Peripheral Arteries. Front Physiol 8, 855 (2017).
33. C. C. Y. Poon, Y. T. Zhang, Y. B. Liu, Modeling of pulse transit time under the effects of hydrostatic pressure for cuffless blood pressure measurements. 2006 3rd Ieee/Embs International Summer School on Medical Devices and Biosensors Doi 10.1109/Issmdbs.2006.360098, 65-+(2006).
34. J. Y. Foo, S. J. Wilson, G. R. Williams, M. A. Harris, D. M. Cooper, Pulse transit time changes observed with different limb positions. Physiol Meas 26, 1093-1102 (2005).
35. R. C. Block et al., Conventional pulse transit times as markers of blood pressure changes in humans. Sci Rep-Uk 10 (2020).
36. Y. Huang et al., Deep inertial poser: Learning to reconstruct human pose from sparse inertial measurements in real time. ACM Transactions on Graphics (TOG) 37, 1-15 (2018).
37. M. Schuster, K. K. Paliwal, Bidirectional recurrent neural networks. IEEE transactions on Signal Processing 45, 2673-2681 (1997).
38. S. Hochreiter, J. Schmidhuber, Long short-term memory. Neural computation 9, 1735-1780 (1997).
39. J. Geissinger, M. M. Alemi, S. E. Change, A. Asbeck, Virginia Tech Natural Motion Dataset. University Libraries, Virginia Tech.
40. S. Raschka, Model evaluation, model selection, and algorithm selection in machine learning. arXiv preprint arXiv: 1811.12808 (2018).
41. B. Efron, The jackknife, the bootstrap and other resampling plans (SIAM, 1982).
42. D. Korteweg, Ueber die Fortpflanzungsgeschwindigkeit des Schalles in elastischen Röhren. Annalen der Physik 241, 525-542 (1878).
43. D. J. Hughes, C. F. Babbs, L. A. Geddes, J. D. Bourland, Measurements of Young's modulus of elasticity of the canine aorta with ultrasound. Ultrason Imaging 1, 356-367 (1979).
44. G. S. Stergiou et al., A Universal Standard for the Validation of Blood Pressure Measuring Devices: Association for the Advancement of Medical Instrumentation/European Society of Hypertension/International Organization for Standardization (AAMI/ESH/ISO) Collaboration Statement. Hypertension 71, 368-374 (2018).
45. P. L. Marino, The ICU book (Lippincott williams & wilkins, 2007).
46. S. Gandhi, W. R. Lewis, “ECG monitoring in the ICU” in Oxford textbook of critical care, A. Webb, D. Angus, S. Finfer, L. Gattinoni, M. Singer, Eds. (Oxford University Press, 2016), chap. 129.
47. R. Lee, “Pulse oximetry and capnography in the ICU” in Oxford textbook of critical care, A. Webb, D. Angus, S. Finfer, L. Gattinoni, M. Singer, Eds. (Oxford University Press, 2016), chap. 73.
48. A. Jubran, Pulse oximetry. Crit Care 19, 272 (2015).
49. H. U. Chung et al., Binodal, wireless epidermal electronic systems with in-sensor analytics for neonatal intensive care. Science 363 (2019).
50. U. Orhan, A. Aydin, Heart Rate Detection on Single-Arm ECG by Using Dual-Median Approach. Arab J Sci Eng 45, 6573-6581 (2020).
51. P. S. Raj, D. Hatzinakos, Feasibility of Single-Arm Single-Lead Ecg Biometrics. Eur Signal Pr Conf, 2525-2529 (2014).
52. Anonymous, IEEE Standard for Wearable, Cuffless Blood Pressure Measuring Devices—Amendment 1. IEEE Std 1708a-2019 (Amendment to IEEE Std 1708-2014) 10.1109/IEEESTD.2019.8859685, 1-35 (2019).
53. A. Paszke et al., Pytorch: An imperative style, high-performance deep learning library. arXiv preprint arXiv: 1912.01703 (2019).
54. J. H. Geissinger, A. T. Asbeck, Motion Inference Using Sparse Inertial Sensors, Self-Supervised Learning, and a New Dataset of Unscripted Human Motion. Sensors (Basel) 20 (2020).
55. I. Loshchilov, F. Hutter, Decoupled weight decay regularization. arXiv preprint arXiv: 1711.05101 (2017).
56. M. H. N. van Velzen, A. J. Loeve, S. P. Niehof, E. G. Mik, Increasing accuracy of pulse transit time measurements by automated elimination of distorted photoplethysmography waves. Med Biol Eng Comput 55, 1989-2000 (2017).
57. T. G. Papaioannou et al., Mean arterial pressure values calculated using seven different methods and their associations with target organ deterioration in a single-center study of 1878 individuals. Hypertension Research 39, 640-647 (2016).

What is claimed is:

1. A cuffless blood pressure monitor, comprising:
a signal acquisition element including a set of sensors that generate data responsive to transmural and relative external pressure;
the sensors including at least two of a barometer, gyroscope, and an accelerometer;
a processor configured to track altitude and calculate relative external pressure responsively to signals from two or more of said barometer, said gyroscope, and said accelerometer, and output said relative external pressure; and
said processor configured to calculate a transmural pressure responsively to a signal from at least one pulse wave sensor based on the relative external pressure.

2. The monitor of claim 1, further comprising the at least one pulse wave sensor, wherein the at least one pulse wave sensor includes at least one plethysmograph sensor and at least one of (a) a second plethysmograph sensor, wherein the two plethysmograph sensors can be used to measure pulse transit time or pulse wave velocity, and (b) a sensor that detects heartbeat that can be used to estimate pulse transit time or pulse wave velocity.

3. The monitor of claim 1, further comprising a magnetometer wherein said processor is configured to track altitude and calculate relative external pressure responsively to signals from said magnetometer as well as said barometer, gyroscope, and accelerometer.

4. A cuffless blood pressure monitor, comprising:
a device support that can be worn over an artery;
the device support having a pulse wave detection element, an external-pressure processing element, a blood pressure tracking processing element, a calibration processing element, and a stability processing element, wherein said stability processing element is configured to detect periods of stable blood pressure;
the pulse wave detection element including at least one plethysmographic sensor which outputs a wave form;
the external-pressure processing element including a processor to estimate external pressure from both of a contact pressure sensor for measuring contact pressure when applied to a user and a hydrostatic pressure sensor that includes two or more of an accelerometer, a gyroscope, and a barometer, wherein the external-pressure processing element is configured to combine signals from the two or more of an accelerometer, a gyroscope, a barometer to track altitude changes in real-time.

5. The monitor of claim 4, wherein the external-pressure processing element includes the hydrostatic pressure sensor and is configured to combine signals from the two or more of an accelerometer, a gyroscope, a barometer, with signals from a magnetometer to track the altitude changes in real-time.

6. The monitor of claim 5, wherein the hydrostatic pressure sensor includes all three of an accelerometer, a gyroscope, and a barometer.

7. The monitor of claim 4, wherein the at least one plethysmographic sensor is one plethysmographic sensor and wherein the pulse wave detection element also includes a sensor that detects a subject's heartbeat.

8. The monitor of claim 4, wherein a relationship between blood pressure and the signals from the sensors is obtained by an analytical algorithm, a linear regression, a polynomial regression, machine learning, or a combination thereof.

9. The monitor of claim 8, wherein the blood pressure and said sensors are related by monitoring the change in external pressure over a predefined period of time and the effect on the signals acquired by the sensors such that blood pressure is constant over the predefined period of time so that the calibration processing element can calculate parameters needed to fit or update the algorithm used for blood pressure tracking.

10. The monitor of claim 9, wherein said relationship between blood pressure and said sensors is obtained when the stability processing element indicates blood pressure is constant over said predefined period of time.

11. The monitor of claim 9, wherein a calibration is automatically begun in response to a change in external pressure and the calibration processing element outputs instructions on a display indicating steps for a user-assisted calibration.

12. The monitor of claim 4, wherein,
the shape of the wave form is used to obtain pulse wave velocity, transmural pressure, or blood pressure using an empirical algorithm, and.
the controller is configured to output a signal indicating an estimate of blood pressure.

13. A blood pressure monitor, comprising:
a set of sensors including at least an accelerometer and a pulse wave sensor; and
a processor configured to track arm orientation based on signals from the accelerometer and, based on the tracked arm orientation, calculate a transmural pressure based on signals from the pulse wave sensor.

14. The monitor of claim 13, further comprising a signal acquisition element including a set of sensors that generate data responsive to transmural and relative external pressure.

15. The monitor of claim 13, wherein the set of sensors further includes at least one of a gyroscope and a magnetometer.

16. The monitor of claim 13, wherein the processor is configured to track arm orientation using a trained deep neural network.

17. The monitor of claim 16, wherein the trained deep neural network comprises at least one bi-directional Long Short-Term Memory (LSTM) layer.

18. The monitor of claim 13, wherein the processor is configured to track arm orientation by feeding output signals from the set of sensors into an Unscented Kalman Filter.

19. The monitor of claim 13, wherein the set of sensors includes a time-of-flight sensor.

20. The monitor of claim 13, wherein a person specific calibration is performed to configure the processor to track the arm orientation.

21. The monitor of claim 13, wherein the processor is configured to calculate a transmural pressure by computing a transmural pressure error based on the tracked arm orientation and compensating for the transmural pressure error.

22. The monitor of claim 13, wherein the processor is configured to track arm orientation based on signals from the accelerometer, wherein the accelerometer is located at a user's wrist.
